(19) United States
(12) Patent Application Publication
(10) Pub. No.: US 2021/0065845 A1
(43) Pub. Date: Mar. 4, 2021

(54) SCORING VARIANTS IN AN EXOME TO PREDICT AN EFFECT OF THE VARIANTS ON GENE FUNCTION

(71) Applicant: Tata Consultancy Services Limited, Mumbai (IN)

(72) Inventors: Sutapa DATTA, Hyderabad (IN); Rajgopal Srinivasan, Hyderabad (IN); Vinay Lanke, Hyderabad (IN)

(73) Assignee: Tata Consultancy Services Limited, Mumbai (IN)

(21) Appl. No.: 16/990,464

(22) Filed: Aug. 11, 2020

(30) Foreign Application Priority Data
Aug. 27, 2019 (IN) 201921034531

(51) Int. Cl. G16B 20/20; C12Q 1/6811; G16B 40/00; G16B 50/00

(52) U.S. Cl. CPC G16B 20/20; G16B 50/00; G16B 40/00; C12Q 1/6811

(57) ABSTRACT

The present disclosure is generally relates to technique for scoring variants to evaluate an effect of the variants on gene function. The present system and method assigns scores for the plurality of variants that are occurred in a particular transcript corresponding to a protein coding gene comprised in the exome. The plurality of variants including the synonymous variants, the non-synonymous variants, the frameshift indels and the non-frameshift indels, the variants that spans into a coding exonic intronic boundary region, and the splice site variants, considering an interplay between a pair of alleles in order to understand as to what extent the variant may impact the gene, based on number of risk alleles present in the gene. The final score of the variant indicate probable effect of the variant, higher the score more will be the effect of the variant on gene.

### PRIORITY CLAIM

This U.S. patent application claims priority under 35 U.S.C. § 119 to: Indian Patent Application No. 201921034531, filed on 27 Aug. 2019. The entire contents of the aforementioned application are incorporated herein by reference.

### TECHNICAL FIELD

The disclosure herein generally relates to techniques for analyzing genomic variants, and, more particularly, to a method and a system for scoring variants in an individual exome to predict an effect of the variants on the gene function.

### BACKGROUND

Decades of genetic research has identified several biomarkers. Specifically in last decade, genetic research has advanced with introduction of high throughput sequencing technology and thereby generating an enormous amount of genomic data, providing molecular insights into several unprecedented human genetic variations and their relation to diseases. An individual human exome may contain millions of variants, specially, single nucleotide variants (SNVs) and indels, out of which only some variants may have an impact on a gene function. So an accurate prediction of an effect of the variants play a major role in determining adverse effect on the gene function and overall health condition of the individual.

However identifying causal variants having a risk on the gene function, from the millions of variants present in the individual exome is really a challenging task. Annotating the variants to determine functional consequences of the variants still remain a complex task due to difficulty in interpreting the variants. Several machine learning based variant scoring techniques have been proposed in the art to detect pathogenicity of the variants. Conventional variant scoring techniques have been extensively used in clinical genomics and research to determine likely consequences of the variants on the gene function based on the detected pathogenicity.

However, the conventional variant scoring techniques have considered non-synonymous variants as they are predicted as more pathogenic, but some of synonymous variants may be pathogenic and cause diseases. Frameshift indels are one of the most deleterious mutation as they may cause complete loss of function of the gene. But there may be several frameshift indels in the gene that may compensate each other and thereby prevent complete loss of function of the corresponding gene. The conventional variant scoring techniques have limitations with the compensating variants to score the frameshift indels. Mutations in splice site region may disrupt a splicing mechanism completely. Similarly, branch points plays an important role in the splicing mechanism and the mutations in the branch point may have the adverse effect on the splicing mechanism. However the conventional variant scoring techniques have limitations to deal with splice site variants that spans into a coding exonic and intronic boundary region, and the branch point mutations, while scoring the variants to predict the effect of the variants on the gene function.

### SUMMARY

Embodiments of the present disclosure present technological improvements as solutions to one or more of the above-mentioned technical problems recognized by the inventors in conventional systems.

In an aspect, there is provided a processor-implemented method for scoring variants in an exome to predict an effect of the variants on gene function, the method comprising the steps of: receiving, via the one or more hardware processors, a dataset comprising a plurality of variants corresponding to the exome, wherein the plurality of variants are one or more single nucleotide variants (SNVs) and one or more indels; annotating, via the one or more hardware processors, each of the plurality of variants comprised in the dataset with corresponding variant information, to form a plurality of annotated variants; identifying, via the one or more hardware processors, one or more variants, out of the plurality of annotated variants, occurring in a transcript of a plurality of transcripts corresponding to a protein coding gene comprised in the exome, to form a set of variants, wherein the one or more variants are identified based on a corresponding transcript ID; separating, via the one or more hardware processors, variants in a Y-chromosome from the set of variants, to form a revised set of variants; identifying, via the one or more hardware processors, (i) one or more SNVs present in the coding exonic region and a coding intronic region, and one or more indels present in the coding intronic region, based on a corresponding minor allele frequency (MAF) value, and (ii) one or more indels present in a coding exonic region, from the revised set of variants, to form a subset of variants; assessing, via the one or more hardware processors, the identified one or more SNVs and the identified one or more indels from the subset of variants, wherein assessing the identified one or more SNVs comprises (i) selecting the one or more SNVs based on a corresponding ethnicity wise allele frequency (ETH_AF) value, from the identified one or more SNVs, and (ii) assigning a score for each of the selected one or more SNVs, based on (i) presence in the coding exonic region and (ii) presence in the coding intronic region, and wherein assessing the identified one or more indels comprises assigning the score for each of the identified one or more indels, based on (i) presence in a coding exonic intronic boundary region (ii) presence in the coding exonic region, and (iii) presence in the coding intronic region; assigning, via the one or more hardware processors, a final score for each of the selected one or more SNVs and the identified one or more indels, based on the corresponding assigned score, a corresponding genomic evolutionary rate profiling (Gerp)++ RSbase value and a corresponding sub-region residual variation intolerance scores (SubRVIS) value; and predicting, via the one or more hardware processors, the effect of the one or more variants on the gene function, based on the corresponding final score, corresponding genotype information and hapoinsufficiency of the gene.

In another aspect, there is provided a system for masking and unmasking of sensitive data, the system comprising: a memory storing instructions; one or more communication interfaces; and one or more hardware processors coupled to the memory via the one or more communication interfaces, wherein the one or more hardware processors are configured by the instructions to: receive a dataset comprising a plurality of variants corresponding to the exome, wherein the plurality of variants are one or more single nucleotide variants (SNVs) and one or more indels; annotate each of the plurality of variants comprised in the dataset with corresponding variant information, to form a plurality of annotated variants; identify one or more variants, out of the plurality of annotated variants, occurring in a transcript of a plurality of transcripts corresponding to a protein coding gene comprised in the exome, to form a set of variants, wherein the one or more variants are identified based on a corresponding transcript ID; separate variants in a Y-chromosome from the set of variants, to form a revised set of variants; identify (i) one or more SNVs present in the coding exonic region and a coding intronic region, and one or more indels present in the coding intronic region, based on a corresponding minor allele frequency (MAF) value, and (ii) one or more indels present in a coding exonic region, from the revised set of variants, to form a subset of variants; assess the identified one or more SNVs and the identified one or more indels from the subset of variants, wherein assessing the identified one or more SNVs comprises (i) selecting the one or more SNVs based on a corresponding ethnicity wise allele frequency (ETH_AF) value, from the identified one or more SNVs, and (ii) assigning a score for each of the selected one or more SNVs, based on (i) presence in the coding exonic region and (ii) presence in the coding intronic region, and wherein assessing the identified one or more indels comprises assigning the score for each of the identified one or more indels, based on (i) presence in a coding exonic intronic boundary region (ii) presence in the coding exonic region, and (iii) presence in the coding intronic region; assign a final score for each of the selected one or more SNVs and the identified one or more indels, based on the corresponding assigned score, a corresponding genomic evolutionary rate profiling (Gerp)++ RSbase value and a corresponding sub-region residual variation intolerance scores (SubRVIS) value; and predict the effect of the one or more variants on the gene function, based on the corresponding final score, corresponding genotype information and haploinsufficiency of the gene.

In yet another aspect, there is provided a computer program product comprising a non-transitory computer readable medium having a computer readable program embodied therein, wherein the computer readable program, when executed on a computing device, causes the computing device to: receive a dataset comprising a plurality of variants corresponding to the exome, wherein the plurality of variants are one or more single nucleotide variants (SNVs) and one or more indels; annotate each of the plurality of variants comprised in the dataset with corresponding variant information, to form a plurality of annotated variants; identify one or more variants, out of the plurality of annotated variants, occurring in a transcript of a plurality of transcripts corresponding to a protein coding gene comprised in the exome, to form a set of variants, wherein the one or more variants are identified based on a corresponding transcript ID; separate variants in a Y-chromosome from the set of variants, to form a revised set of variants; identify (i) one or more SNVs present in the coding exonic region and a coding intronic region, and one or more indels present in the coding intronic region, based on a corresponding minor allele frequency (MAF) value, and (ii) one or more indels present in a coding exonic region, from the revised set of variants, to form a subset of variants; assess the identified one or more SNVs and the identified one or more indels from the subset of variants, wherein assessing the identified one or more SNVs comprises (i) selecting the one or more SNVs based on a corresponding ethnicity wise allele frequency (ETH_AF) value, from the identified one or more SNVs, and (ii) assigning a score for each of the selected one or more SNVs, based on (i) presence in the coding exonic region and (ii) presence in the coding intronic region, and wherein assessing the identified one or more indels comprises assigning the score for each of the identified one or more indels, based on (i) presence in a coding exonic intronic boundary region (ii) presence in the coding exonic region, and (iii) presence in the coding intronic region; assign a final score for each of the selected one or more SNVs and the identified one or more indels, based on the corresponding assigned score, a corresponding genomic evolutionary rate profiling (Gerp)++ RSbase value and a corresponding sub-region residual variation intolerance scores (SubRVIS) value; and predict the effect of the one or more variants on the gene function, based on the corresponding final score, corresponding genotype information and haploinsufficiency of the gene.

In an embodiment of the present disclosure, each variant of the plurality of variants comprises a corresponding chromosome number, a corresponding genomic position, a corresponding reference allele, a corresponding alternative allele, and the corresponding genotype information.

In an embodiment of the present disclosure, the corresponding variant information of each variant of the plurality of variants comprising one or more of: a corresponding gene name, the corresponding subRVIS value, the corresponding minor allele frequency (MAF) value, the corresponding ethnicity wise allele frequency (ETH_AF) value, a corresponding region of the variant, the corresponding transcript ID, a corresponding mutation type, corresponding information related to change in amino-acid, the corresponding Gerp++ RSbase value, the corresponding dbScSNV values comprising a corresponding adaboost (Ada) value and a corresponding random forest (RF) value, a corresponding deleterious annotation of genetic variants using neural networks (DANN) value, a corresponding sorting intolerant from tolerant (SIFT) value, a corresponding protein variation effect analyzer (PROVEAN) value, a corresponding functional analysis through hidden markov models (FATHMM) value, a corresponding mendelian clinically applicable pathogenicity (M-CAP) value, and a corresponding meta-analytic support vector machine (MetaSVM) value.

In an embodiment of the present disclosure, assigning the score for each of the selected one or more SNVs present in the coding exonic region, comprising: categorizing the selected one or more SNVs into: (i) coding exonic splice region SNVs and (ii) coding exonic non-splice region SNVs, wherein the coding exonic splice region SNVs are the selected one or more SNVs that fall under a splice region and the coding exonic non-splice region SNVs are the selected one or more SNVs that does not fall under the splice region; assigning an initial score to the coding exonic non-splice region SNVs; assigning initial scores to the coding exonic splice region SNVs, based on the corresponding Ada value and the corresponding RF value; sub-categorizing the coding exonic splice region SNVs and the coding exonic non-splice region SNVs into: (i) non-synonymous SNVs group (ii) synonymous SNVs group and (iii) gain-loss mutation SNVs group, based on the corresponding mutation type, wherein the gain-loss mutation SNVs group includes stop gain mutation SNVs, stop loss mutation SNVs, start gain mutation SNVs and start loss mutation SNVs; assigning the score for each of the coding exonic splice region SNVs and each of the coding exonic non-splice region SNVs, comprised in the non-synonymous SNVs group, based on (i) the corresponding initial score, (ii) outcome of SNVs deleteriousness prediction tools, and (iii) a change in amino acid within predefined amino acid groups and an outcome of SNVs protein function effect prediction tool; assigning the score for each of the coding exonic splice region SNVs and each of the coding exonic non-splice region SNVs, comprised in the synonymous SNVs group, based on (i) the corresponding initial score and (ii) the outcome of SNVs deleteriousness prediction tool; and assigning the score for each of the coding exonic splice region SNVs and each of the coding exonic non-splice region SNVs, comprised in the gain-loss mutation SNVs group, based on (i) the corresponding initial score and (ii) the outcome of SNVs deleteriousness prediction tool.

In an embodiment of the present disclosure, assigning the score for each of the identified one or more indels present in the coding exonic region, comprising: categorizing the identified one or more indels present in the coding exonic region into (i) a non-frameshift indels group and (ii) a frameshift indels group, based on the corresponding mutation type; assigning the score for each of the identified one or more indels comprised in the non-frameshift indels group, based on (i) the corresponding MAF value (ii) the corresponding ETH_AF value and (iii) the outcome of indels deleteriousness prediction tool; and assigning the score for each of the identified one or more indels comprised in the frameshift indels group, comprising: categorizing the identified one or more indels into one or more deletion indels and one or more insertion indels, based on a length of the corresponding reference allele (len_ref) and a length of the corresponding altered allele (len_alt); calculating an insertion length of each of the one or more insertion indels and a deletion length (del_len) of each of the one or more deletion indels, based on the corresponding len_ref and the corresponding len_alt; calculating a haplo1_indel value as a sum of insertions occurring in haplotype1 (haplo1_ins value) and deletions occurring in haplotype1 (haplo1_del value), and a haplo2_indel value as sum of the insertions occurring in haplotype2 (haplo2_ins value) and the deletions occurring in haplotype2 (haplo2_del value), haplotype1 (h1) represent one gene copy and haplotype2 (h2) represent the another gene copy, wherein the haplo1_ins value is a total length of the one or more insertion indels present in the haplotype1 (h1), the haplo1_del value is a total length of the one or more deletion indels present in the haplotype1 (h1), and the haplo2_ins value is a total length of the one or more insertion indels present in the haplotype2 (h2), the haplo2_del value is a total length of the one or more deletion indels present in the haplotype2 (h2); calculating a haplotype1_score based on a change in reading frame of the gene in haplotype1 (h1) and a h1_count and a haplotype2_score based on a change in reading frame of the gene in haplotype2 (h2) and a h2_count, wherein the h1_count is calculated based on a number of indels present in the haplotype1 (h1) and the number of indels present in the haplotype1 (h1) having the MAF value greater than the predefined Th_MAF value, and the h2_count is calculated based on the number of indels present in the haplotype2 (h2) and the number of indels present in the haplotype2 (h2) having the MAF value greater than the predefined Th_MAF value; and assigning the score for each of the identified one or more indels based on a h1_allele score and a h2_allele score, wherein the h1_allele score is calculated based on the haplotype1_score and the h1_count, and the h2_allele score is calculated based on the haplotype2_score and the h2_count.

In an embodiment of the present disclosure, assigning the score for each of the identified one or more indels present in the coding exonic intronic boundary region, comprising: selecting the one or more indels from the identified one or more indels, based on the corresponding MAF value less than the predefined threshold value; categorizing the selected one or more indels into insertion indels and deletion indels, based on a length of the corresponding reference allele (len_ref) and a length of the corresponding altered allele (len_alt); sub-categorizing the insertion indels into donor insertion indels and acceptor insertion indels, and the deletion indels into donor deletion indels and acceptor deletion indels, based on the corresponding genomic position; assigning the score for each of the donor deletion indels, by: calculating a MaxEnt value for a plurality of donor consensus (GTs) present between −50 bp and +50 bp from a position of the corresponding donor deletion indel to identify the donor consensus having the maximum MaxEnt value from the plurality of donor consensus (GTs); and assigning the score for the corresponding donor deletion indel based on a change in a exon length, considering the identified donor consensus having the maximum MaxEnt value as a cryptic donor GT; assigning the score for each of the acceptor deletion indels, by: calculating the MaxEnt value for a plurality of acceptor consensus (AGs) present between −50 bp and +50 bp from the position of the corresponding acceptor deletion indel to identify the acceptor consensus having the maximum MaxEnt value from the plurality of the acceptor consensus (AGs); and assigning the score for the corresponding acceptor deletion indel based on the change in the exon length, considering the identified acceptor consensus having the maximum MaxEnt value as a cryptic acceptor AG; assigning the score for each of the donor insertion indels based on: (i) the corresponding donor insertion indel generating or not generating a new donor consensus, (ii) the MaxEnt value of the new donor consensus and the MaxEnt value of the natural donor consensus in mutated sequence, and (iii) the MaxEnt value of the new donor consensus, the MaxEnt value of the natural donor consensus in wildtype sequence and the change in the exon length; and assigning the score for each of the acceptor insertion indels based on: (i) the corresponding acceptor insertion indel generating or not generating a new acceptor consensus, (ii) the MaxEnt value of the new acceptor consensus and the MaxEnt value of the natural acceptor consensus in mutated sequence, and (iii) the MaxEnt value of the new acceptor consensus, the MaxEnt value of the natural acceptor consensus in wildtype sequence and the change in the exon length.

In an embodiment of the present disclosure, assigning the score for each of the identified one or more indels and the selected one or more SNVs present in the coding intronic region, comprising: categorizing the identified one or more indels and the selected one or more SNVs present in the coding intronic region into (i) donor coding intronic variants and (ii) acceptor coding intronic variants, based on the corresponding genomic position; assigning the score for each of the donor coding intronic variants and the acceptor coding intronic variants, wherein, assigning the score for each of the donor coding intronic variants, based on: (i) the variant having a natural donor site disrupted or weakened or not affected (ii) the MaxEnt value of the natural donor site, if the variant with natural donor site not disrupted, (iii) the MaxEnt value of the cryptic donor site, if the cryptic donor site is generated, and (iv) a position of natural donor site and the position of the cryptic donor site; assigning the score for each of the acceptor coding intronic variants, based on the corresponding position of the variant (pos_var) from the acceptor site, wherein: assigning the score for each of the acceptor coding intronic variants having the pos_var less than 15, based on: (i) the variant with the natural acceptor site disrupted or weakened or not affected, (ii) the MaxEnt value of the natural acceptor site, if the variant with natural acceptor site not disrupted, (iii) the MaxEnt value of the cryptic acceptor site, if the cryptic acceptor site is generated, and (iv) a position of natural acceptor site and the position of the cryptic acceptor site; assigning the score for each of the acceptor coding intronic variants having the pos_var between 15 and 20, based on: (i) the variant causing the branch point disruption, and (ii) the variant not causing the branch point disruption, wherein, the score for the variant causing the branch point disruption is assigned based on a presence of an existing compensating branch point or a newly created compensating branch point; and the score for the variant not causing the branch point disruption is assigned based on at least one of (i) the natural acceptor site weakened or not weakened (ii) the MaxEnt value of natural acceptor site, (iii) the MaxEnt value of cryptic acceptor site if the cryptic acceptor site is generated (iv) the position of natural acceptor site and the position of the cryptic acceptor site; assigning the score for each of the acceptor coding intronic variants having the pos_var between 21 and 49, based on at least one of: (i) branch point disrupted or not disrupted (ii) presence of an existing compensating branch point (iii) a newly created branch point; and assigning the score for each of the acceptor coding intronic variants having the pos_var 50 or more, with the predefined value.

It is to be understood that both the foregoing general description and the following detailed description are exemplary and explanatory only and are not restrictive of the invention, as claimed.

### BRIEF DESCRIPTION OF THE DRAWINGS

The accompanying drawings, which are incorporated in and constitute a part of this disclosure, illustrate exemplary embodiments and, together with the description, serve to explain the disclosed principles:

FIG. 1 is a functional block diagram of a system for scoring variants in an exome to predict an effect of the variants on gene, in accordance with an embodiment of the present disclosure.

FIG. 2A and FIG. 2B illustrate flow diagrams of a processor implemented method using the system of FIG. 1 for scoring variants in an exome to predict an effect of the variants on gene, in accordance with an embodiment of the present disclosure.

FIG. 3A through FIG. 3P illustrate flow diagrams of a processor implemented method using the system of FIG. 1 for scoring each variant type and based on a corresponding region, in accordance with an embodiment of the present disclosure.

FIG. 4 depicts a receiver operating curve (ROC) showing prediction performance of a method for scoring variants in an exome to predict an effect of the variants on gene, using a Clinvar database, in accordance with an embodiment of the present disclosure.

FIG. 5 depicts a receiver operating curve (ROC) using a deleterious annotation of genetic variants using neural networks (DANN) value corresponding to the variants present in a Clinvar database, in accordance with an embodiment of the present disclosure.

FIG. 6 depicts a receiver operating curve (ROC) using a functional analysis through hidden markov models (FATHMM) value corresponding to the non-synonymous variants present in a Clinvar database, in accordance with an embodiment of the present disclosure.

### DETAILED DESCRIPTION OF EMBODIMENTS

Exemplary embodiments are described with reference to the accompanying drawings. In the figures, the left-most digit(s) of a reference number identifies the figure in which the reference number first appears. Wherever convenient, the same reference numbers are used throughout the drawings to refer to the same or like parts. While examples and features of disclosed principles are described herein, modifications, adaptations, and other implementations are possible without departing from the scope of the disclosed embodiments. It is intended that the following detailed description be considered as exemplary only, with the true scope being indicated by the following claims.

Gene variants in general are classified into SNVs (single nucleotide variants) and indels (insertion variants and deletion variants). The SNVs are the variants with a change in single nucleotide at a particular position whereas the indels are the variants with either addition or deletion of nucleotides in the particular position. The SNVs may further be classified into coding exonic SNVs and coding intronic SNVs, based on a region of the SNV. The coding exonic SNVs are further classified into synonymous SNVs, non-synonymous SNVs and gain-loss mutation SNVs, based on a type of change in amino acid. The synonymous SNVs are the SNVs where the change in a nucleotide does not change corresponding amino acid whereas the non-synonymous SNVs cause change in the amino acid. The gain-loss mutation SNVs are the SNVs where the change in a nucleotide causes stop loss, stop gain, start loss and start gain.

The indels may further be classified into coding exonic indels, coding intronic indels, coding exonic-intronic boundary indels and splice site indels depending on the region of the gene. The coding exonic indels are further classified as frameshift (FS) indels and non-frame shift (NFS) indels depending on number of inserted or deleted nucleotides. The FS indels are more deleterious compared to the NFS indels, which cause complete loss of function of the gene due to the change in reading frame of the gene as number of inserted or deleted nucleotides are not multiple of three, whereas the NFS indels inserts or deletes the sequences, where the length of which is multiple of three causing no disruption to the reading frame.

The coding intronic indels are further classified as donor site indels and acceptor site indels depending on the positon of the indels. A donor site indel occurs near 5′ end of an intron whereas acceptor site indel occurs near 3′ end of the intron. The splice site indels are the variants that changes the nucleotides of donor site (GT) or acceptor site (AG) while the variants occurring at the boundary of the donor site and corresponding preceding exon or the acceptor site and corresponding succeeding exon are called coding exonic-intronic boundary indels.

In accordance with the present disclosure, the method assigns scores for the plurality of variants that are occurring in a particular transcript corresponding to a protein coding gene comprised in the individual exome, to predict the effect of the variants on the gene function. The plurality of variants including the synonymous variants, the non-synonymous variants, the gain-loss mutations, the frameshift indels and the non-frameshift indels, the variants that spans into a coding exonic intronic boundary region, and the splice site variants. An interplay between a pair of alleles is considered to understand as to what extent the variant may impact the gene function, based on number of risk alleles present in the gene.

In accordance with the present disclosure, the method receives a plurality of variants and selects one or more variants from the plurality of variants to get a set of variants based on criteria such as minor allele frequency (MAF), region of variants, chromosome number, type of gene, genotype, and so on. Next the one or more variants from the set of variants are assigned with the scores based on the annotation information and utilizing an existing biological knowledge. The variants are given a high score that are thought to be deleterious based on the annotation information such as the region of variants, the type of mutation, and prediction outcome of several existing prediction tools such as a deleterious annotation of genetic variants using neural networks (DANN), a functional analysis through hidden markov models (FATHMM), a meta-analytic support vector machine (MetaSVM), a protein variation effect analyzer (PROVEAN), MaxEnt and so on. The non-synonymous SNVs are given high score as compared to the synonymous SNVs because the non-synonymous SNVs are likely to be more deleterious than the synonymous SNVs as the non-synonymous SNVs changes corresponding amino acid in a protein sequence. A final score of each variant indicate probable effect of the variant, higher the final score more will be the effect of the variant on the gene function.

In accordance with the present disclosure, the method for scoring variants in the exome to predict the effect of the variants on gene, assigns numeric score to each variant in the range 1 to 10, leading to the final score of each variant in the range ‘−2 to +8’ to provide an estimate of the deleteriousness of the corresponding variant. However the provided ranges are exemplary and not limited to the scope of the invention. It may be understood to the person skilled in the art that, the scores may be assigned with different ranges and scales, by implementing the disclosed method.

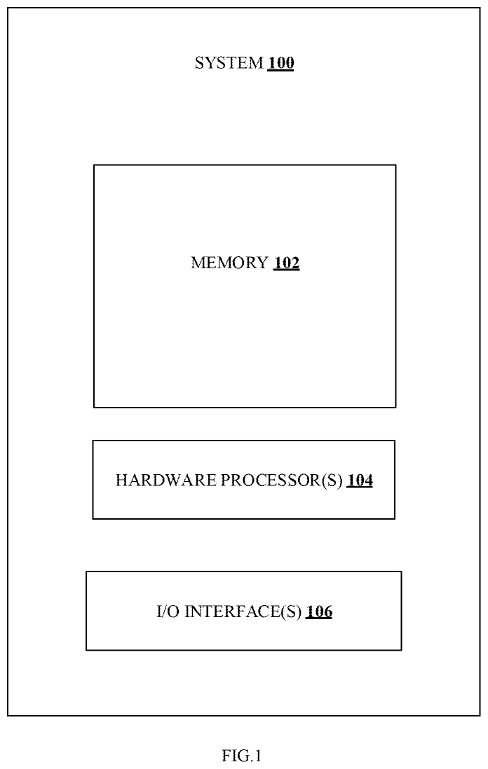

Referring now to the drawings, and more particularly to FIG. 1 through FIG. 6, where similar reference characters denote corresponding features consistently throughout the figures, there are shown preferred embodiments and these embodiments are described in the context of the following exemplary systems and/or methods.

FIG. 1 is a functional block diagram of a system for scoring variants in an exome to predict an effect of the variants on gene, in accordance with an embodiment of the present disclosure. In an embodiment, the system 100 includes one or more processors 104, communication interface device(s) or input/output (I/O) interface(s) 106, and one or more data storage devices or memory 102 operatively coupled to the one or more processors 104. The one or more processors 104 may be hardware processors and can be implemented as one or more microprocessors, microcomputers, microcontrollers, digital signal processors, central processing units, state machines, graphics controllers, logic circuitries, and/or any devices that manipulate signals based on operational instructions. Among other capabilities, the processor(s) are configured to fetch and execute computer-readable instructions stored in the memory.

The I/O interface(s) 106 can include a variety of software and hardware interfaces, for example, a web interface, a graphical user interface, and the like and can facilitate multiple communications within a wide variety of networks N/W and protocol types, including wired networks, for example, LAN, cable, etc., and wireless networks, such as WLAN, cellular, or satellite. In an embodiment, the I/O interface(s) can include one or more ports for connecting a number of devices to one another or to another server.

The memory 102 may include any computer-readable medium known in the art including, for example, volatile memory, such as static random access memory (SRAM) and dynamic random access memory (DRAM), and/or non-volatile memory, such as read only memory (ROM), erasable programmable ROM, flash memories, hard disks, optical disks, and magnetic tapes.

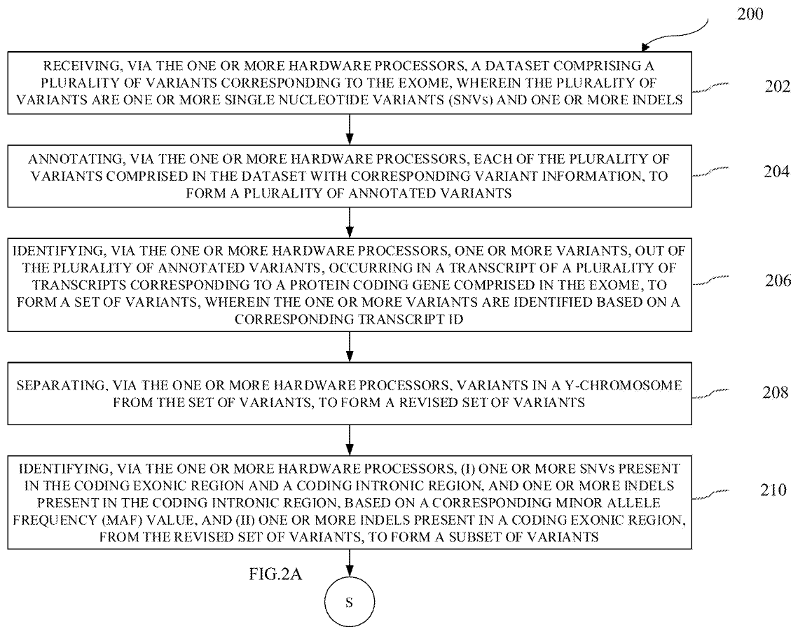

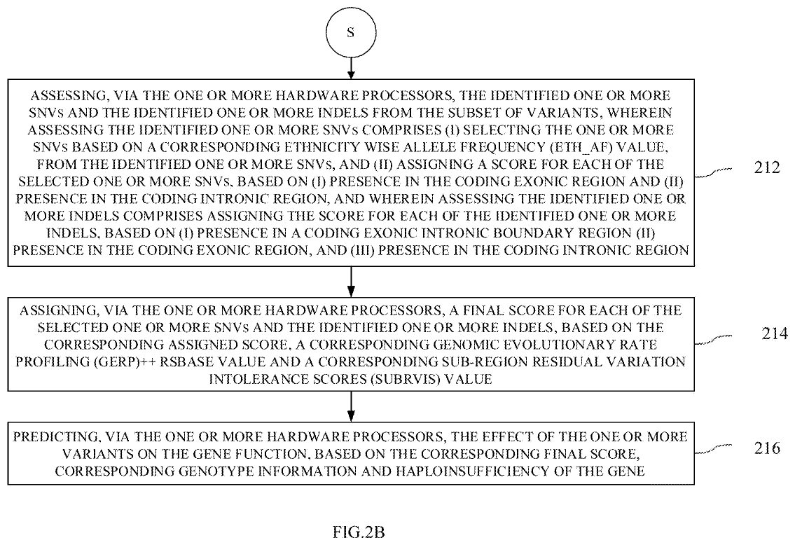

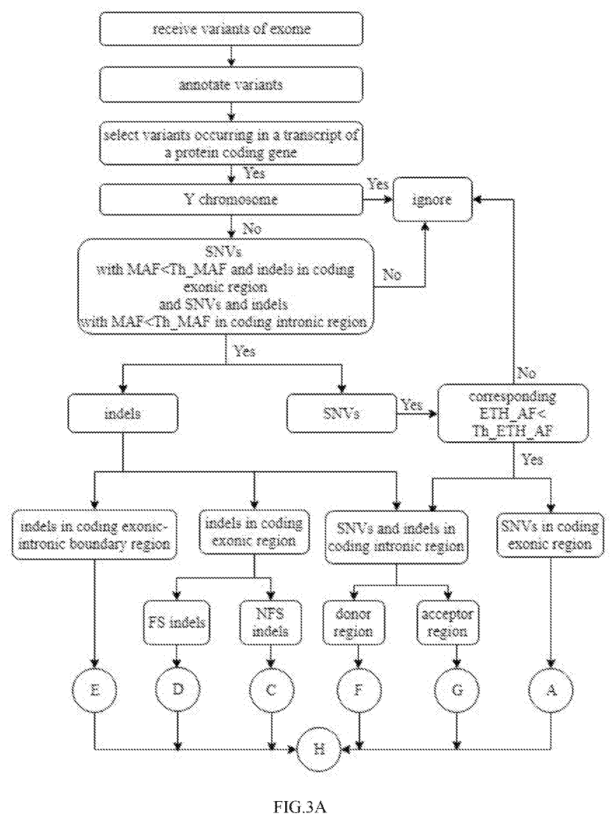

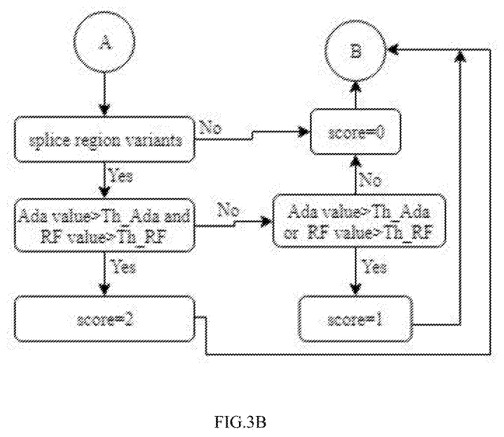

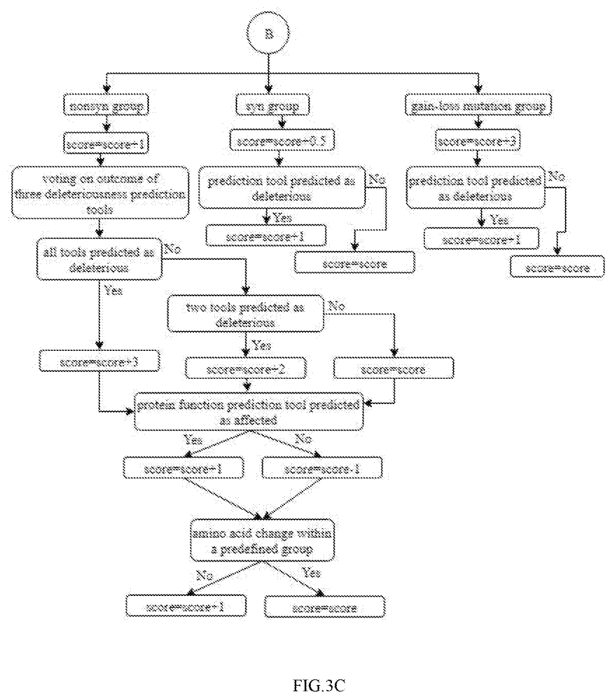

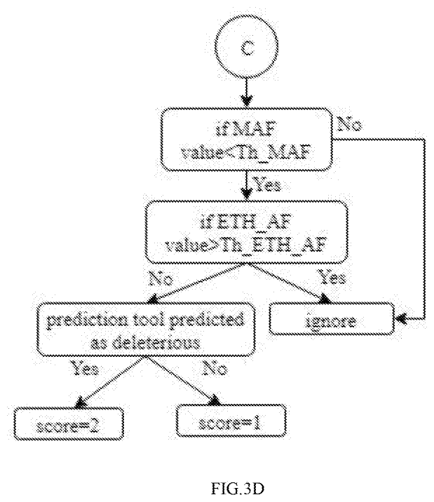

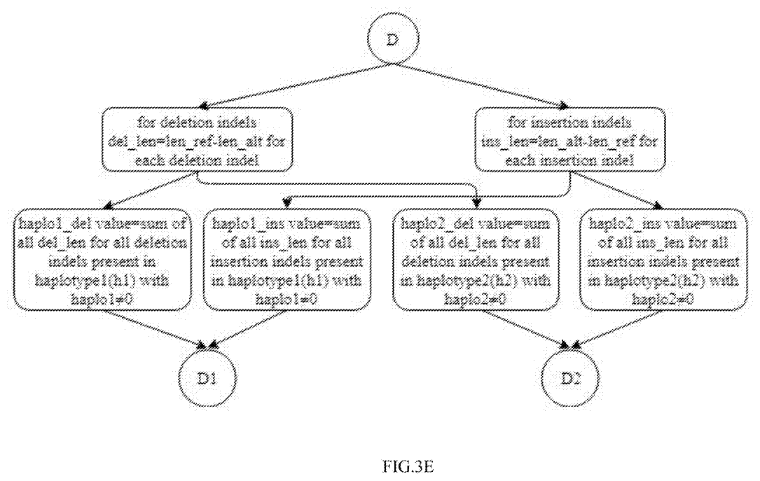

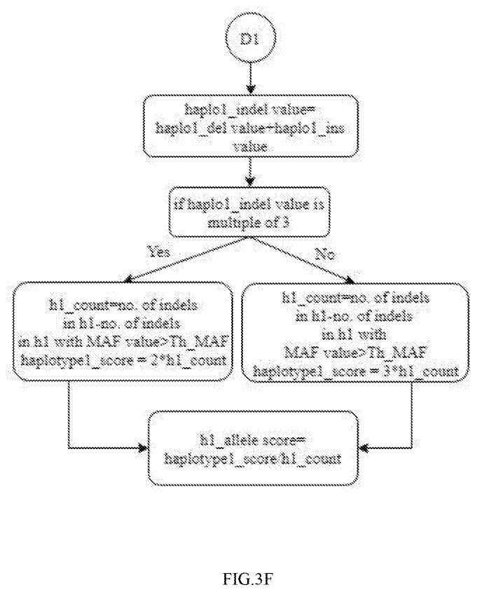

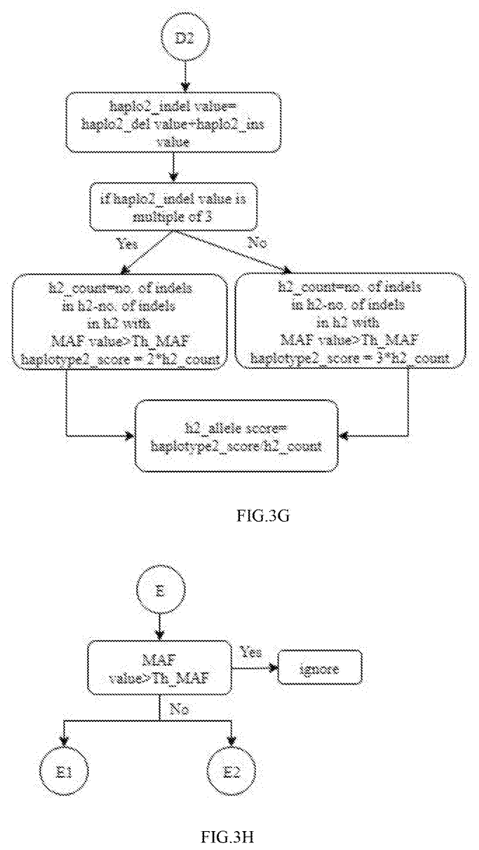

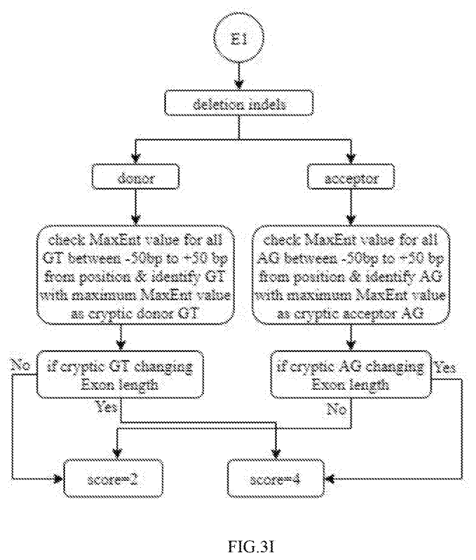

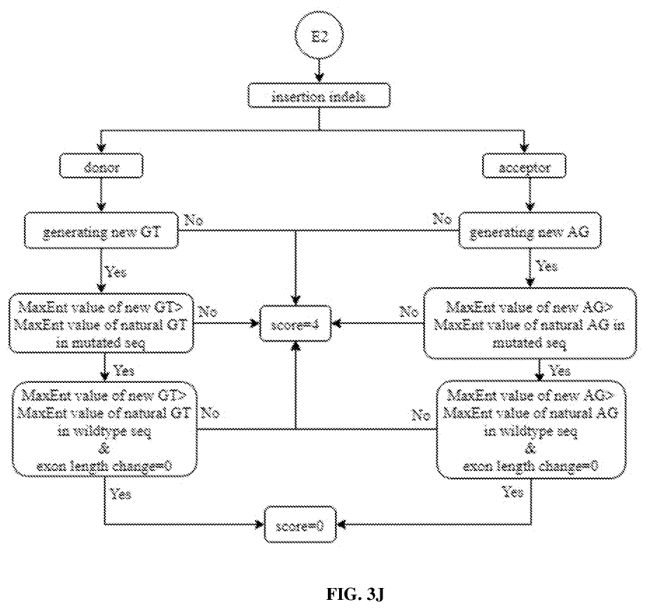

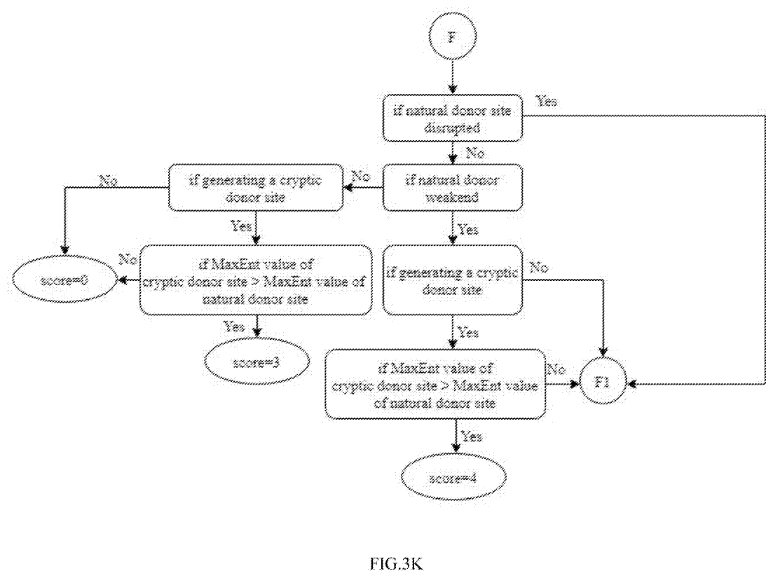

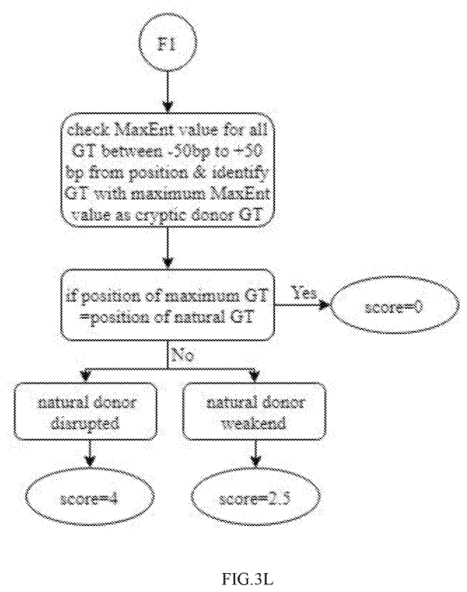

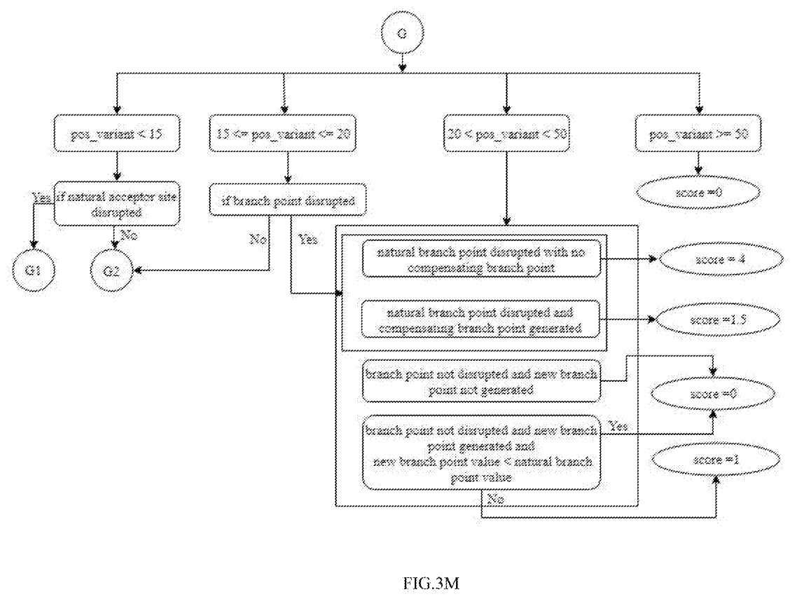

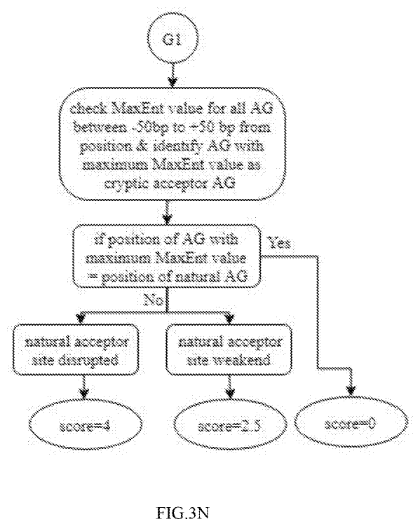

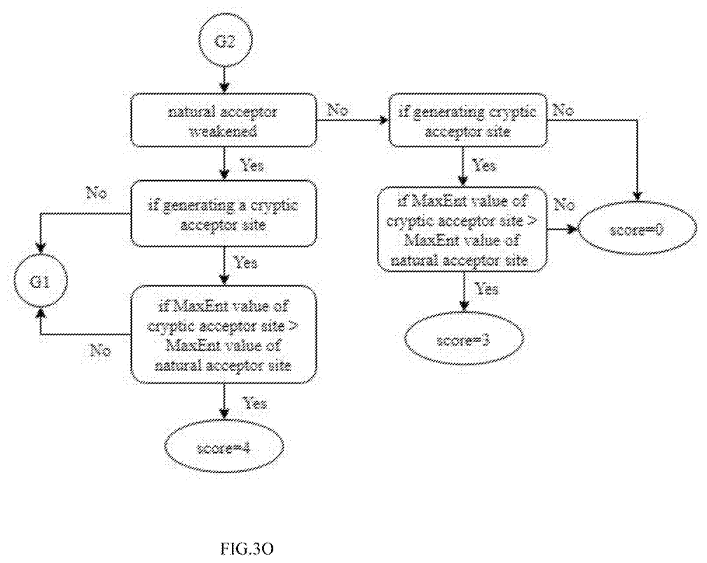

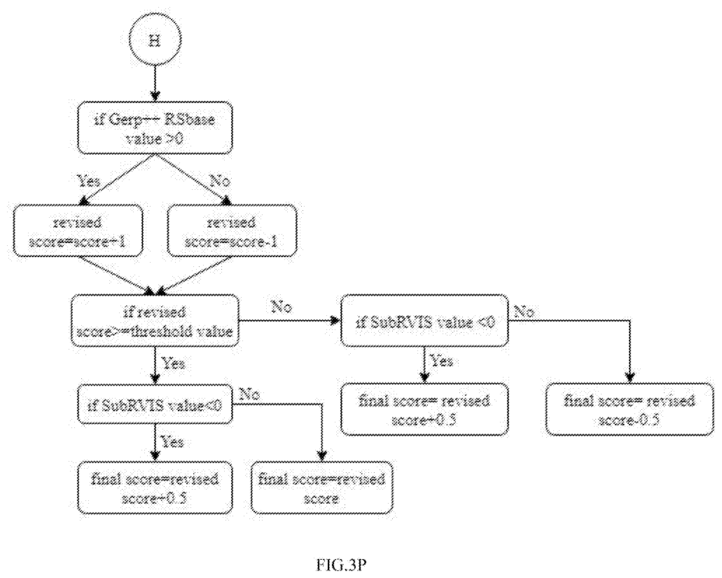

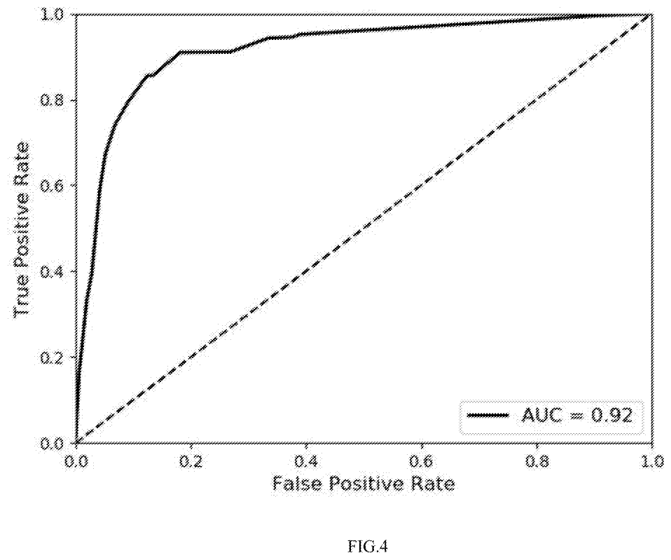

FIG. 2A and FIG. 2B illustrate flow diagrams of a processor implemented method 200 using the system 100 of FIG. 1 for scoring variants in an exome to predict an effect of the variants on gene, in accordance with an embodiment of the present disclosure. FIG. 3A through FIG. 3P illustrate flow diagrams of a processor implemented method 200 using the system 100 of FIG. 1 for scoring each variant type and based on the corresponding region, in accordance with an embodiment of the present disclosure. The steps of the method 200 will now be explained in detail with reference to the system 100. Although process steps, method steps, techniques or the like may be described in a sequential order, such processes, methods and techniques may be configured to work in alternate orders. In other words, any sequence or order of steps that may be described does not necessarily indicate a requirement that the steps be performed in that order. The steps of processes described herein may be performed in any order practical. Further, some steps may be performed simultaneously.

In accordance with an embodiment of the present disclosure, the one or more hardware processors 104 of FIG. 1 are configured to receive at step 202, a dataset comprising a plurality of variants corresponding to the exome. The dataset may be obtained from publicly available databases such as 1000 genome project, EXAC database etc., and may be in the form of a VCF (variant calling format) file. Each of the plurality of variants includes the corresponding chromosome number, a corresponding genomic position, a corresponding reference allele, a corresponding alternative allele, and the corresponding genotype information.

In accordance with an embodiment of the present disclosure, the one or more hardware processors 104 of FIG. 1 are configured to annotate each of the plurality of variants comprised in the dataset, with corresponding variant information, at step 204, to form a plurality of annotated variants. In an embodiment, the corresponding variant information of each of the plurality of variants including one or more of: the corresponding gene name, the corresponding subRVIS value, the corresponding MAF value, the corresponding ethnicity wise allele frequency (ETH_AF) value, a corresponding region of the variant, the corresponding transcript ID, a corresponding mutation type, corresponding information related to change in amino-acid, the corresponding Gerp++ RSbase value, corresponding dbScSNV values comprising a corresponding adaboost (Ada) value and a corresponding random forest (RF) value, a corresponding deleterious annotation of genetic variants using neural networks (DANN) value, a corresponding sorting intolerant from tolerant (SIFT) value, a corresponding protein variation effect analyzer (PROVEAN) value, a corresponding functional analysis through hidden markov models (FATHMM) value, a corresponding mendelian clinically applicable pathogenicity (M-CAP) value, and a corresponding meta-analytic support vector machine (MetaSVM) value.

In an embodiment, each of the plurality of variants is annotated by tagging the corresponding variant information, using a tool such as Varant which provide different type of annotations in form of categories, utilizing several databases such as RefGene, Regulomedb, UTRdb, spliceDB, dbSNP, 1000Genome and so on. For example, a variant identity and frequency category provides the MAF value for each variant. Similarly, an experimentally defined genomic features category provides the gene name, the region of the variant, the transcript ID, the mutation type, the information related to change in amino-acid, where the region of the variant includes an exon region, a intron region, a untranslated region (utr) or intergenic region where the variant is occurring and the mutation type comprising the non-synonymous SNVs, the synonymous SNVs, the frameshift indels, the non-frameshift indels, stop gain, stop loss, start gain or start loss mutations.

Every gene comprises two alleles present in a heterozygous state (two alleles are different in both copies of the gene) or a homozygous state (two alleles are same in the both copies of the gene). The major allele is the most common allele and minor allele is the less common allele in a particular population. The corresponding MAF value of the variant is the frequency at which a minor allele occurs in the population. The more the MAF value is, the more common the corresponding variant is in the population. Some alleles may be more common or specific to the particular population. The corresponding ETH_AF value of the variant is the allele frequency that occurs in a particular ethnic group of the population.

The dbScSNV values are pre-computed prediction values for the SNVs that may occur in the splice region, obtained from a dbscSNV database. The pre-computed prediction values suggest an indication of whether the SNV is expected to affect a splicing of the gene. The dbScSNV values comprises two values for each SNV occurring in the splice region, namely the adaboost (Ada) value and the random forest (RF) value. The Ada value is obtained based on the adaboost method whereas the RF value is obtained based on the random forest (RF) method. Both the Ada value and the RF value are scaled from 0 to 1, where higher value indicate a greater probability that the SNV may alter the splicing of the gene.

The deleterious annotation of genetic variants using neural networks (DANN) value is obtained using the DANN tool where the DANN value is used to measure the deleteriousness of the SNVs present in the genome in order to effectively prioritize the causal variants in genetic analyses. The DANN value ranges between 0 and 1. A SNV with higher DANN value indicate that the corresponding SNV is predicted to be deleterious. Typically, the SNVs with the corresponding DANN value more than 0.9 are predicted to be deleterious.

The FATHMM value is obtained using the functional analysis through hidden markov models (FATHMM) tool which is a hidden markov model based method to find the deleteriousness of the missense variants. FATHMM pred values are defined based on the FATHMM value. If the FATHMM value is less than or equal to ‘−1.5’, then the FATHMM pred value is D indicating that the variant is predicted as Damaging (D), otherwise the FATHMM pred value is T indicating that the variant is predicted as Tolerated (T).

The PROVEAN value is obtained using the protein variation effect analyzer (PROVEAN) tool, which predicts functional effects of protein sequence variations for SNVs and non-frameshift indels. The PROVEAN value ranges from −14 to 14. The smaller the PROVEAN value, the more likely the variant has damaging effect. PROVEAN pred values are defined based on the PROVEAN value. Typically, if the PROVEAN value is less than or equal to ‘−2.5’, then the PROVEAN pred value is D indicating that the variant is predicted as Damaging (D), otherwise the PROVEAN pred value is N indicating that the variant is predicted as Neutral (N).

The SIFT value is obtained using the sorting intolerant from tolerant (SIFT) tool which is used to predict whether the amino acid substitution affects the corresponding protein function. The SIFT value ranges between 0 and 1. The smaller the SIFT value, the more likely the variant has damaging effect. SIFT pred values are defined based on the SIFT value. If the SIFT value is less than ‘0.05’, then the SIFT pred value is D indicating that the variant is predicted as Damaging (D), otherwise the SIFT pred value is T indicating that the variant is predicted as Tolerated (T).

The genomic evolutionary rate profiling (Gerp)++ RSbase value is used to identify sites under evolutionary constraint and represent nucleotide level constraint score within deep multiple sequence alignments. The GERP++ uses a significantly faster and more statistically robust maximum likelihood estimation procedure in order to identify constrained elements. The higher the Gerp++ RSbase value, the more conserved the site is.

A meta-analytic support vector machine (MetaSVM) value is generated using a support vector machine based ensemble tool, used to evaluate deleteriousness of the missense mutations. The higher MetaSVM value mean the corresponding variant is more likely to be damaging.

The subRVIS value provide a measure of intolerance of a genic subregion to mutational burden. The higher the subRVIS value the more is the tolerance to mutational burden for the genic sub-region. The subRVIS value is obtained based upon allele frequency as represented in whole exome sequence data from the NHLBI-ESP6500 data set.

A gencode bed file and a human genome sequence version 19 fasta file are used to retrieve reference and alternate sequence information. The fasta file consists of human genome sequence and gencode file consists of positional information of every intron and exon for the available transcripts along with gene annotations. So for calculating MaxEnt values of donor and acceptor site variants according to the present invention, a corresponding sequence from −50 bp to +50 bp region from the position of variant from fasta file is extracted. As the sequence is extracted from reference genome fasta file, it may be represented as a wildtype sequence. A mutated sequence is obtained by replacing the reference sequence with mutated sequence at the position if the variant in the wildtype sequence. A branch point is a sequence with consensus nucleotide “A” occurring within −50 bp to −15 bp upstream of acceptor site that helps in splicing.

In accordance with an embodiment of the present disclosure, the one or more hardware processors 104 of FIG. 1 are configured to identify at step 206, one or more variants out of the plurality of annotated variants, occurring in the particular transcript of plurality of transcripts corresponding to the protein coding gene comprised in the exome, to form a set of variants. In an embodiment, the one or more variants are identified based on the corresponding transcript ID to form the set of variants.

In accordance with an embodiment of the present disclosure, the one or more hardware processors 104 of FIG. 1 are configured to separate the variants in a Y-chromosome from the set of variants at step 208, to form a revised set of variants. The variants in the Y-chromosome are separated through filtration as the variants present only in male and mostly associated with infertility and defect in a male reproductive system. The revised set of variants comprises the set of variants except the variants that are occurring in the Y-chromosome.

In accordance with an embodiment of the present disclosure, the one or more hardware processors 104 of FIG. 1 are configured to identify at step 210, (i) one or more SNVs present in the coding exonic region and a coding intronic region, and one or more indels present in the coding intronic region, based on the corresponding MAF value, and (ii) one or more indels present in a coding exonic region, from the revised set of variants, to form a subset of variants for assessment. In an embodiment, the one or more SNVs present in the coding exonic region and the coding intronic region, and the one or more indels present in the coding intronic region are selected based on the corresponding MAF value less than a predefined MAF threshold value (Th_MAF).

For example, the predefined threshold value (Th_MAF) may be 0.01, which indicates that 1% of a population is having the allele which mean that the variant is quite common in the population and may not cause any adverse effect. The predefined threshold value (Th_MAF) of 0.01 is applied to reduce the number of variants as generally rare variants are associated with the adverse effect.

In accordance with an embodiment of the present disclosure, the one or more hardware processors 104 of FIG. 1 are configured to assess the identified one or more SNVs and the identified one or more indels, at step 212. In an embodiment, assessing the identified one or more SNVs includes (i) selecting the one or more SNVs having the corresponding ETH_AF value less than a predefined threshold (Th_ETH_AF) value, from the identified one or more SNVs, and (ii) assigning a score for each of the selected one or more SNVs, based on (i) presence in the coding exonic region and (ii) presence in the coding intronic region. For example, the predefined threshold (Th_ETH_AF) value used to identify the one or more SNVs is 0.01, which indicate at least 1% of the population of that sample group is having that particular allele in the exome which further mean variant is very common in that ethnic group and may not be associated with any adverse effect.

In an embodiment, assessing the identified one or more indels includes assigning the score for each of the identified one or more indels, based on (i) presence in the coding exonic intronic boundary region (ii) presence in the coding exonic region, and (iii) presence in the coding intronic region. FIG. 3A depicts selecting variants for assigning scores, in accordance with an embodiment of the present disclosure.

In an embodiment, the one or more hardware processors 104 of FIG. 1 are configured to assign the score for each of the selected one or more SNVs present in the coding exonic region, by categorizing the selected one or more SNVs into coding exonic splice region SNVs and coding exonic non-splice region SNVs. The coding exonic splice region SNVs are the selected one or more SNVs that fall under the splice region and the coding exonic non-splice region SNVs are the selected one or more SNVs that does not fall under the splice region.

The coding exonic non-splice region SNVs are assigned with a predefined initial score. In an embodiment, the predefined initial score may be ‘0’. Then, the coding exonic splice region SNVs are assigned with the predefined initial scores, based on the corresponding Ada value and the corresponding RF value. In an embodiment, the predefined initial score ‘2’ is assigned for the SNVs having the corresponding Ada value and the corresponding RF value greater than a predefined Ada threshold (Th_Ada) value and a predefined RF threshold (Th_RF) value respectively. The predefined initial score ‘1’ is assigned for the SNVs having the corresponding Ada value or the corresponding RF value greater than the predefined Th_Ada value and the predefined Th_RF value respectively. The predefined initial score ‘0’ is assigned for the SNVs having the corresponding Ada value or the corresponding RF value lesser than the predefined Th_Ada value and the predefined Th_RF value respectively. In an embodiment, the predefined Th_Ada value may be ‘0.6’ and the predefined Th_RF value may be ‘0.6’. Particularly, FIG. 3B depicts assigning scores for coding exonic splice region SNVs and the coding exonic non-splice region SNVs, in accordance with an embodiment of the present disclosure.

Further, the coding exonic splice region SNVs and the coding exonic non-splice region SNVs are sub-categorized into: (i) non-synonymous SNVs group (ii) synonymous SNVs group and (iii) gain-loss mutation SNVs group, based on the corresponding mutation type. The gain-loss mutation SNVs group includes the SNVs that are stop gain mutation SNVs, stop loss mutation SNVs, start gain mutation SNVs and start loss mutation SNVs. Particularly, FIG. 3C depicts assigning scores for coding exonic splice region SNVs and the coding exonic non-splice region SNVs according to synonymous SNVs group, non-synonymous SNVs group and gain-loss mutation SNVs group, in accordance with an embodiment of the present disclosure.

In an embodiment, the score for each of the coding exonic splice region SNVs and each of the coding exonic non-splice region SNVs comprised in the non-synonymous SNVs group is assigned based on the corresponding predefined initial score, voting on outcome of deleteriousness prediction of the SNVs, a change in amino acid within a predefined amino acid groups, and an effect of the corresponding protein function. In an embodiment, the voting on the deleteriousness prediction of the SNVs is carried out using any three deleteriousness prediction tools from the available deleteriousness prediction tools including the DANN tool, the MetaSVM tool, the FATHMM tool, the M-CAP tool, the PROVEAN tool, a variant effect scoring tool (VEST), a combined annotation dependent depletion (CADD) tool, a rare exome variant ensemble learner (REVEL) tool, and so on. Each tool from the provided list gives the corresponding prediction value from which the deleteriousness of the variant is determined. In an embodiment, the effect of the corresponding protein function is predicted by the SIFT tool with the help of the SIFT value.

If all the three deleteriousness prediction tools indicate that the corresponding SNV is deleterious, the corresponding protein function is affected and has change in the amino acid within the predefined amino acid groups predefined from physio-chemical characteristics, then the score for the corresponding SNV is assigned according to a relation ‘score=initial score+3+1+1’. If no change in the amino acid within the predefined amino acid groups, then the score for the corresponding SNV is assigned according to the relation ‘score=initial score+3+1+1+1’. If the corresponding protein function is not affected and but has change in the amino acid within the predefined amino acid groups, then the score for the corresponding SNV is assigned according to the relation ‘score=initial score+3+1−1’. If no change in the amino acid within the predefined amino acid groups, then the score for the corresponding SNV is assigned according to the relation ‘score=initial score+3+1+1−1’.

If two out of the three deleteriousness prediction tools indicate that the corresponding SNV is deleterious, then if the corresponding protein function is affected and has change in the amino acid within the predefined amino acid groups, then the score for the corresponding SNV is assigned according to the relation ‘score=initial score+2+1+1’. If no change in the amino acid within the predefined amino acid groups, then the score for the corresponding SNV is assigned according to the relation ‘score=initial score+2+1+1+1’. If the corresponding protein function is not affected but has change in the amino acid within the predefined amino acid groups, then the score for the corresponding SNV is assigned according to the relation ‘score=initial score+2+1−1’. If no change in the amino acid within the predefined amino acid groups, then the score for the corresponding SNV is assigned according to the relation ‘score=initial score+2+1+1−1’.

If one out of the three deleteriousness prediction tools indicate that the corresponding SNV is deleterious, then if the corresponding protein function is affected and has change in the amino acid within the predefined amino acid groups, then the score for the corresponding SNV is assigned according to the relation ‘score=initial score+1+1’. If no change in the amino acid within the predefined amino acid groups, then the score for the corresponding SNV is assigned according to the relation ‘score=initial score+1+1+1’. If the corresponding protein function is not affected and but has change in the amino acid within the predefined amino acid groups, then the score for the corresponding SNV is assigned according to the relation ‘score=initial score+1−1’. If no change in the amino acid within the predefined amino acid groups, then the score for the corresponding SNV is assigned according to the relation ‘score=initial score+1+1−1’.

In an embodiment, the predefined amino acid groups are: an acidic and amide group including aspartic acid, glutamic acid, asparagine and glutamine, a basic group including histidine, lysine and arginine, a aliphatic group including glycine, alanine, valine, leucine and isoleucine, an aromatic group including phenylalanine, tyrosine and tryptophan, a cyclic group including proline, and a hydroxyl or sulfur group including serine, cysteine, threonine and methionine. The predefined amino acid groups are formed from amino acids, based on a corresponding structure of the amino acid and general chemical characteristics of corresponding R groups.

In an embodiment, the score for each of the coding exonic splice region variants and each of the coding exonic non-splice region variants comprised in the synonymous SNVs group is assigned based on (i) the corresponding predefined initial score and (ii) the outcome of SNVs deleteriousness prediction tool. In an embodiment, the DANN tool is used as SNVs deleteriousness prediction tool. If the outcome of the DANN tool is deleterious (which is determined through the DANN value), then the score for the corresponding SNV is assigned according to the relation: ‘score=initial score+1+0.5’, else the score for the corresponding SNV is assigned according to the relation: ‘score=initial score+0.5’.

In an embodiment, the score for each of the coding exonic splice region variants and each of the coding exonic non-splice region variants comprised in the gain-loss mutation SNVs group is assigned based on (i) the corresponding predefined initial score and i(ii) the outcome of SNVs deleteriousness prediction tool. The DANN tool is used as SNVs deleteriousness prediction tool. If the outcome of the DANN tool is deleterious (which is determined through the DANN value), then the score for the corresponding SNV is assigned according to the relation: ‘score=initial score+3+1’, else the score for the corresponding SNV is assigned according to the relation: ‘score=initial score+3’.

In an embodiment, the one or more hardware processors 104 of FIG. 1 are configured to assign the score for each of the identified one or more indels present in the coding exonic region, by categorizing the identified one or more indels present in the coding exonic region into (i) a non-frameshift indels group and (ii) a frameshift indels group, based on the corresponding mutation type. In an embodiment, the score for each of the identified one or more indels comprised in the non-frameshift indels group is assigned based on (i) the corresponding MAF value, (ii) the corresponding ETH_AF value and (iii) the outcome of the indels deleteriousness prediction tool. Particularly, FIG. 3D depicts assigning scores for coding exonic non-frameshift indels, in accordance with an embodiment of the present disclosure.

In an embodiment, if the corresponding MAF value of the non-frameshift indel is lesser than the predefined MAF threshold (Th_MAF) value, if the corresponding ETH_AF value of the non-frameshift indel is lesser than the predefined ETH_AF threshold (Th_ETH_AF) value and if the outcome of the corresponding indels deleteriousness prediction toolis deleterious, then the score for the corresponding non-frameshift indel is assigned with ‘2’. If the corresponding MAF value of the non-frameshift indel is lesser than the predefined Th_MAF value, if the corresponding ETH_AF value of the non-frameshift indel is lesser than the predefined Th_ETH_AF value and if the outcome of the indels deleteriousness prediction tool is deleterious, then the score for the corresponding non-frameshift indel is assigned with ‘1’. If the corresponding MAF value of the non-frameshift indel is greater than the predefined Th_MAF value and if the corresponding ETH_AF value of the non-frameshift indel is greater than the predefined Th_ETH_AF value, then such non-frameshift indels are not assigned with any score. In an embodiment, the predefined Th_MAF value is ‘0.01’ and the predefined Th_ETH_AF value is ‘0.01’. In an embodiment, the PROVEAN tool is used as indels deleteriousness prediction tool and the corresponding PROVEAN value is used to determine the deleteriousness of the non-frameshift indels.

In an embodiment, the score for each of the identified one or more indels comprised in the frameshift indels group is assigned by calculating an insertion length (ins_len) in case of insertion indel and a deletion length (del_len) in case of deletion indel, of each of the identified indels comprised in the frameshift indels group occurring in the corresponding gene. The insertion length (ins_len) and the deletion length (del_len) are calculated as a difference between the length of the corresponding reference allele (len_ref) and the length of the corresponding altered allele (len_alt). In an embodiment, if the len_ref is greater than the len_alt, then such indel is identified as deletion indel, and the del_len is calculated according to the relation: del_len=len_ref−len_alt. If the len_ref is lesser than the len_alt, then such indel is identified as insertion indel, and the ins_len is calculated according to the relation: ins_len=len_alt−len_ref.

A haplo1 representing a haploid genotype in one gene copy and a haplo2 representing the haploid genotype in another gene copy are identified. Then a haplo1_indel value is calculated as a sum of insertions (haplo1_ins value) and deletions (haplo1_del value) occurring in one gene copy such as haplotype1. Similarly a haplo2_indel value is calculated as sum of insertions (haplo2_ins value) and deletions (haplo2_del value) occurring in another gene copy such as haplotype2. The haplo1_ins value is the total length of the insertion indels present in the haplotype1 of the gene. The haplo1_del value is the total length of the deletion indels present in the haplotype1 of the gene. The haplo2_ins value is the total length of the insertion indels present in the haplotype2 of the gene. The haplo2_del value is the total length of the deletion indels present in the hapotype2 of the gene.

If the haplo1_indel value is completely divisible by ‘3’, then, a haplotype1_score is calculated according to the relation: haplotype1_score=2*h1_count. If the haplo1_indel value is not completely divisible by ‘3’, then, the haplotype1_score is calculated according to the relation: haplotype1_score=3*h1_count. The h1_count is calculated as a difference between a number of indels present in haplotype1 and the number of indels present in haplotype1 having the MAF value greater than the predefined MAF threshold (Th_MAF) value. In an embodiment, the predefined Th_MAF value is ‘0.01’. A h1_allele score is calculated according to the relation: h1_allele score=haplotype1_score/h1_count. The haplo1_indel value is completely divisible by ‘3’ indicates that there is no change in the reading frame of the gene.

Similarly, if the haplo2_indel value is completely divisible by ‘3’, then, a haplotype2_score is calculated according to the relation: haplotype2_score=2*h2_count. If the haplo2_indel value is not completely divisible by ‘3’, then, the haplotype2_score is calculated according to the relation: haplotype2_score=3*h2_count. The h2_count is calculated as a difference between a number of indels present in haplotype2 and the number of indels present in haplotype2 having the MAF value greater than the predefined Th_MAF value. In an embodiment, the predefined Th_MAF value is ‘0.01’. A h2_allele score is calculated according to the relation: h2_allele score=haplotype2_score/h2_count. The haplo2_indel value is completely divisible by ‘3’ indicates that there is no change in the reading frame of the gene.

If the frameshift indel is present in the haplotype1, then the score of the corresponding frameshift indel is assigned with the h1_allele score. If the frameshift indel is present in the haplotype2, then the score of the corresponding frameshift indel is assigned with the h2_allele score. Particularly, FIG. 3E through FIG. 3G depicts assigning scores for coding exonic frameshift indels, in accordance with an embodiment of the present disclosure.

In an embodiment, the one or more hardware processors 104 of FIG. 1 are configured to assign the score for each of the identified one or more indels present in the coding exonic intronic boundary region, by selecting the one or more indels having the corresponding MAF value lesser than the predefined Th_MAF value, from the identified one or more indels. The one or more indels having the corresponding MAF value greater than the predefined Th_MAF value are not assigned with any score. In an embodiment, the predefined MAF threshold value is ‘0.01’. Particularly, FIG. 3H through FIG. 3J depicts assigning scores for variants present in coding exonic intronic boundary region, in accordance with an embodiment of the present disclosure.

In an embodiment, the one or more selected indels having the corresponding MAF value lesser than the predefined Th_MAF value are categorized into insertion indels and deletion indels, based on the length of the corresponding reference allele (len_ref) and the length of the corresponding altered allele (len_alt).

The deletion indels are sub-categorized into donor deletion indels and acceptor deletion indels, based on the corresponding genomic position. A MaxEnt value for a plurality of donor consensus (GT) present between −50 bp and +50 bp from a position of the corresponding donor deletion indel from the donor deletion indels, is calculated to identify the donor consensus having the maximum MaxEnt value from the plurality of donor consensus (GT). Similarly, the MaxEnt value for a plurality of acceptor consensus (AG) present between −50 bp and +50 bp from the position of the corresponding acceptor deletion indel from the acceptor deletion indels, is calculated to identify the acceptor consensus having the maximum MaxEnt value from the plurality of the acceptor consensus (AG). The score of the corresponding donor deletion indel is assigned based on a change in the exon length, considering the identified donor consensus having the maximum MaxEnt value as a cryptic donor GT. The exon length change is determined based on a position of the cryptic donor GT and the position of the natural donor GT. Similarly, the score of the corresponding acceptor deletion indel is assigned based on the change in the exon length, considering the identified acceptor consensus having the maximum MaxEnt value as a cryptic acceptor AG. The exon length change is determined based on a position of the cryptic acceptor AG and the position of the natural acceptor AG.

In an embodiment, the score for the corresponding donor deletion indel is assigned with ‘4’, if the position of the identified donor consensus having the maximum MaxEnt value, is not equal to the position of the natural donor consensus (causing a change in the exon length). The score for the corresponding donor deletion indel is assigned with ‘2’, if the position of the identified donor consensus having the maximum MaxEnt value, is equal to the position of the natural donor consensus (not causing a change in the exon length). Similarly, the score for the corresponding acceptor deletion indel is assigned with ‘4’, if the position of the identified acceptor consensus having the maximum MaxEnt value, is not equal to the position of the natural acceptor consensus (causing a change in the exon length). The score for the corresponding acceptor deletion indel is assigned with ‘2’, if the position of the identified acceptor consensus having the maximum MaxEnt value, is equal to the position of the natural acceptor consensus (not causing a change in the exon length).

Similarly, the insertion indels are sub-categorized into donor insertion indels and acceptor insertion indels, based on the corresponding genomic position. The score for each of the donor insertion indels is assigned based on: (i) the corresponding donor insertion indel generating or not generating a new donor consensus, (ii) the MaxEnt value of the new donor consensus and the MaxEnt value of the natural donor consensus in mutated sequence, and (iii) the MaxEnt value of the new donor consensus, the MaxEnt value of the natural donor consensus in wildtype sequence and the change in the exon length.

In an embodiment, if the corresponding donor insertion indel is not generating a new donor consensus, then the score for the corresponding donor insertion indel is assigned with ‘4’. If the corresponding donor insertion indel is generating the new donor consensus but the MaxEnt value of the new donor consensus is lesser than the MaxEnt value of the natural donor consensus in mutated sequence, then the score for the corresponding donor insertion indel is assigned with ‘4’. If the MaxEnt value of the new donor consensus is greater than the MaxEnt value of the natural donor consensus in mutated sequence, but the MaxEnt value of the new donor consensus is lesser than the MaxEnt value of the natural donor consensus in wildtype sequence and there is change in the corresponding exon length, then the score for the corresponding donor insertion indel is assigned with ‘4’. If the MaxEnt value of the new donor consensus is greater than the MaxEnt value of the natural donor consensus in wildtype sequence and there is no change in the corresponding exon length, then the score for the corresponding donor insertion indel is assigned with ‘0’.

Similarly, the score for each of the acceptor insertion indels is assigned based on: (i) the corresponding acceptor insertion indel generating or not generating a new acceptor consensus, (ii) the MaxEnt value of the new acceptor consensus and the MaxEnt value of the natural acceptor consensus in mutated sequence, and (iii) the MaxEnt value of the new acceptor consensus, the MaxEnt value of the natural acceptor consensus in wildtype sequence and the change in the exon length.

In an embodiment, if the corresponding acceptor insertion indel is not generating a new acceptor consensus, then the score for the corresponding acceptor insertion indel is assigned with ‘4’. If the corresponding acceptor insertion indel is generating the new acceptor consensus but the MaxEnt value of the new acceptor consensus is lesser than the MaxEnt value of the natural acceptor consensus in mutated sequence, then the score for the corresponding acceptor insertion indel is assigned with ‘4’. If the MaxEnt value of the new acceptor consensus is greater than the MaxEnt value of the natural acceptor consensus in mutated sequence, but the MaxEnt value of the new acceptor consensus is lesser than the MaxEnt value of the natural acceptor consensus in wildtype sequence and there is a change in the corresponding exon length, then the score for the corresponding acceptor insertion indel is assigned with ‘4’. If the MaxEnt value of the new acceptor consensus is greater than the MaxEnt value of the natural acceptor consensus in wildtype sequence and there is no change in the corresponding exon length, then the score for the corresponding acceptor insertion indel is assigned with ‘0’.

In an embodiment, the one or more hardware processors 104 of FIG. 1 are configured to assign the score for each of the identified one or more indels and the selected one or more SNVs present in the coding intronic region, by categorizing the identified one or more indels and the selected one or more SNVs present in the coding intronic region into (i) donor coding intronic variants and (ii) acceptor coding intronic variants, based on the corresponding genomic position.

The donor coding intronic variants are sub-categorized into (i) disrupted or weakened natural donor site group and (ii) non-disrupted and non-weakened natural donor site group. The disrupted or weakened natural donor site group comprises the donor coding intronic variants having the natural donor site disrupted or weakened. The non-disrupted and non-weakened natural donor site group comprises the donor coding intronic variants having the natural donor site not disrupted and not weakened. Disruption of natural donor site may occur when position of variant is same as the position of natural donor consensus GT. Weakening of natural donor site is decided based on the MaxEnt value of natural donor site in wildtype sequence and MaxEnt value of natural donor in mutated sequence. Particularly, FIG. 3K and FIG. 3L depicts assigning scores for coding intronic variants occurring near a donor site, in accordance with an embodiment of the present disclosure.

In an embodiment, if the variant comprised in the non-disrupted and non-weakened natural donor site group is not having an ability to generate a cryptic donor site, then the score for the corresponding variant is assigned with ‘0’. If the variant comprised in the non-disrupted and non-weakened natural donor site group has the ability to generate the cryptic donor site, and if a cryptic donor site value is lesser than a natural donor site value, then the score for the corresponding variant is assigned with ‘0’. If the variant comprised in the non-disrupted and non-weakened natural donor site group has the ability to generate the cryptic donor site, and if the cryptic donor site value is greater than the natural donor site value, then the score for the corresponding variant is assigned with ‘3’.

In an embodiment, if the variant comprised in the disrupted or weakened natural donor site group, has the ability to generate the cryptic donor site, and if the cryptic donor site value is greater than the natural donor site value, then the score for the corresponding variant is assigned with ‘4’. The MaxEnt value for the plurality of the donor consensus present between −50 bp and +50 bp from the position of the corresponding variant comprised in the disrupted or weakened natural donor site group having (i) the variants whose natural donor site is disrupted (ii)) the variants whose natural donor site is weakened and unable to generate the cryptic donor site, and (iii) the variants whose natural donor site is weakened and has the ability to generate the cryptic donor site but the cryptic donor site value is lesser than the natural donor site value, is calculated to identify the donor consensus having the maximum MaxEnt value. The score for the corresponding variant is then assigned based on (i) a position of the identified donor consensus having the maximum MaxEnt value, (ii) a position of a natural donor consensus and (iii) a corresponding natural donor site disrupted or weakened. In an embodiment, the score for the corresponding variant is assigned with ‘0’, if the position of the identified donor consensus having the maximum MaxEnt value is the same as that of the position of the natural donor consensus. If the position of the identified donor consensus having the maximum MaxEnt value is not the same as that of the position of the natural donor consensus, then the score for the variant is assigned with ‘4’, if the natural donor site of the corresponding variant is disrupted. If the position of the identified donor consensus having the maximum MaxEnt value is not the same as that of the position of the natural donor consensus, then the score for the variant is assigned with ‘2.5’, if the natural donor site of the corresponding variant is weakened.

In an embodiment, the score for the variant comprised in the acceptor coding intronic variants is assigned based on the corresponding position of the variant (pos_var) from the acceptor site. Particularly, FIG. 3M through FIG. 30 depicts assigning scores for coding intronic variants occurring near an acceptor site and a branch point, in accordance with an embodiment of the present disclosure.

In an embodiment, the score for the variant is assigned with ‘0’, if the corresponding position of the variant (pos_var) is more or equal to ‘50’ from the acceptor site. If the corresponding position of the variant (pos_var) is between ‘21’ and ‘49’ from the acceptor site, then the score is assigned with ‘4’, if the natural branch point of the corresponding variant is disrupted and not having a compensating branch point. The score of the variant is assigned with ‘1.5’, if natural branch point is disrupted and a compensated branch point is generated by the corresponding variant. The score for the variant is assigned with ‘0’, if natural branch point is not disrupted and the variant is generating a new branch point and new branch point value is lesser than natural branch point value. The score of the variant is assigned with ‘1’ if natural branch point is not disrupted and variant is generating a new branch point and new branch point value is greater than natural branch point value.

In an embodiment, the branch point value is calculated based on a position weight matrix (PWM) of size 10×4 generated by aligning Mercer's experimentally determined 59,359 human branch sites (10 mers) with branch point consensus nucleotide ‘A’ at 7th position. The alignment was used to calculate the frequency of each nucleotide at each position. The frequency was converted to log odds scores, using the calculated distribution of the four bases in introns as the background frequency. Based on the branch site values obtained from the known branch sites with ‘A’ as branch point and by considering top 75% values in the interquartile range, a threshold value of 1.46 was considered for classifying a site based on branch point value to be a high confidence branch site. Now the branch point value is calculated by taking each 10 mer sequence and calculating the sum of log odd score for each nucleotide corresponding to the 10 mer sequence from PWM. If the branch point value is more than threshold value, then the 7th nucleotide of the 10 mer sequence is considered as branch point.

For the variants whose corresponding position (pos_var) is lesser than ‘15’ from the acceptor site, then the score for the corresponding variant is assigned based on whether the natural acceptor site is disrupted and/or weakened. If the natural acceptor site is not disrupted and not weakened, then if the corresponding variant is not generating a cryptic acceptor site, then the score for the corresponding variant is assigned with ‘0’. Even the corresponding variant has the ability to generate the cryptic acceptor site but the cryptic acceptor site value is lesser than that of natural acceptor site value, then the score for the corresponding variant is assigned with ‘0’. If the cryptic acceptor site value is greater than that of natural acceptor site value, then the score for the corresponding variant is assigned with ‘3’. If the natural acceptor site is weakened but not disrupted, then if the corresponding variant has the ability to generate the cryptic acceptor site, then the score for the corresponding variant is assigned with ‘4’, if the cryptic acceptor site value is greater than that of natural acceptor site value.

The MaxEnt value for the plurality of the acceptor consensus present between −50 bp and +50 bp from the position of the corresponding variant: (i) whose natural acceptor site is disrupted (ii) whose natural acceptor site is weakened but unable to generate a cryptic acceptor site and (iii) whose natural acceptor site is weakened and has the ability to generate the cryptic acceptor site, but the cryptic acceptor site value is lesser than that of the natural acceptor site, to identify the acceptor consensus having the maximum MaxEnt value. If the position of identified acceptor consensus having the maximum MaxEnt value is same as that of the position of the natural acceptor consensus, then the score for the corresponding variant is assigned with ‘0’. If the position of identified acceptor consensus having the maximum MaxEnt value is not same as that of the position of the natural acceptor consensus, then the score for the corresponding variant is assigned with ‘4’ whose natural acceptor site is disrupted, else the score for the corresponding variant is assigned with ‘2.5’ whose natural acceptor site is weakened.

For the variants whose corresponding position (pos_var) is greater than or equal to ‘15’ and lesser than or equal to ‘20’, from the acceptor site, then the score for the corresponding variant is assigned based on whether the branch point is disrupted or not. If the branch point of the corresponding variant is disrupted then the score for the corresponding variant is based on the presence or absence of the compensating branch point. If the branch point of the corresponding variant is not disrupted then the score for the corresponding variant is based on the natural acceptor is weakened or not weakened.

In accordance with an embodiment of the present disclosure, the one or more hardware processors 104 of FIG. 1 are configured to assign a final score for each of the selected one or more SNVs and the identified one or more indels, at step 214, based on the corresponding assigned score, the corresponding Gerp++ RSbase value and the corresponding SubRVIS value. Particularly, FIG. 3P depicts assigning final scores to variants, in accordance with an embodiment of the present disclosure.

In an embodiment, if the corresponding Gerp++ RSbase value of the SNV from the selected one or more SNVs or the indel from the identified one or more indels, is more than zero, then a revised score for the corresponding SNV or indel is assigned according to the relation: ‘revised score=assigned score+1’, else the revised score for the corresponding SNV or indel is assigned according to the relation: ‘revised score=assigned score-1’. If the revised score is greater than or equal to the threshold value and if the corresponding SubRVIS value is less than zero, then the final score for the corresponding SNV or indel is assigned according to the relation: ‘final score=revised score+0.5’, else the final score for the corresponding SNV or indel is assigned according to the relation: ‘final score=revised score’. If the revised score is lesser than the threshold value and if the corresponding SubRVIS value is less than zero, then the final score for the corresponding SNV or indel is assigned according to the relation: ‘final score=revised score+0.5’, else the final score for the corresponding SNV or indel is assigned according to the relation: ‘final score=revised score−0.5’. In an embodiment, the threshold value may be ‘2.5’.

In accordance with an embodiment of the present disclosure, the one or more hardware processors 104 of FIG. 1 are configured to predict the effect of the one or more variants on the gene, at step 216, based on the corresponding final score, the corresponding genotype and the haploinsufficiency of the gene.

A single functional copy may not produce sufficient gene product to carry out the gene function, if the gene is haploinsufficient. The genotype provides state of the variant whether is in heterozygous state or homozygous state or in trans with other variants i.e. if one copy of the gene is damaged or both the copy of the gene is affected. If the variant with high final score is present in both copy of the gene or the variant is in trans with another variant with high final score or if the variant with high final score is present in one copy of the gene and the gene is haploinsufficient, then the corresponding variant may be damaging to the gene function.

In accordance with the present disclosure, all the type of the variants present in the gene are considered while scoring, including the non-synonymous variants, the synonymous variants, the frameshift indels, the non-frameshift indels, the stoploss mutations, the stopgain mutations, the startloss mutations and the start gain mutations as well as mutations occurring in the splice site region. Hence adverse effect of the variants on the gene function is predicted and the variants that may damage the gene function are estimated accurately.

Also, the method 200 assigns the scores to the variants transcript wise, considering the region of variants, the mutation type, the change in the amino acid, as the region of the variant, the mutation type, and the change in amino acid may be different for different transcripts. Hence the effect of variant is estimated transcript wise and may differ with different transcripts.

Further, the method 200 considers all compensating variants haplotype wise for scoring frameshift indels present in the gene. The frameshift indels are generally deleterious but in a particular gene, several frameshift indels may be present compensating with each other and ultimately leading to less deleterious non-frameshift indels. The method 200 predict probable effect of the variants on the gene, considering all the risk alleles present in that gene and haploinsufficiency of the gene, beside predicting the deleteriousness of the variant based on the corresponding final score.

### Experimental Results

To predict the deleterious effect of the variant on the gene function, the threshold value used at step 214 of the method 200, to assign the final score, was determined by assigning scores to the variants present in Clinvar database, except for the frameshift indels which have been assigned with the final score as ‘3’ directly. The pathogenic and likely pathogenic variants are considered as positive data and benign, likely benign variants are considered as negative data. The three deleteriousness prediction tools used for predicting the deleteriousness of coding exonic non-splice region SNVs comprised in the non-synonymous SNVs group, are the DANN tool, the MetaSVM tool and the FATHMM tool.

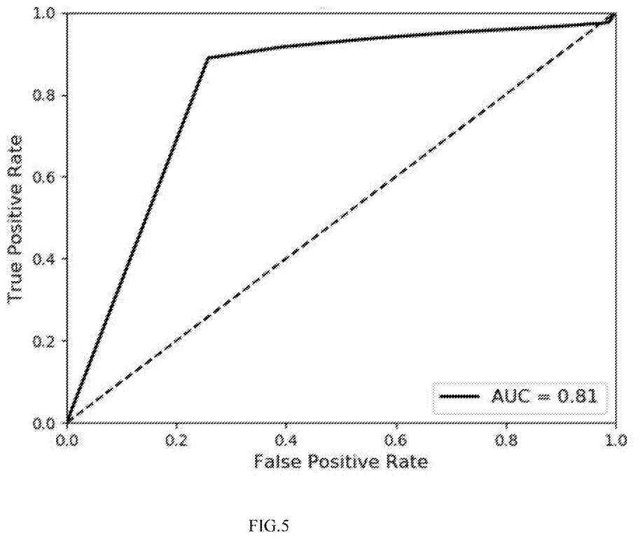

A receiver operating curve (ROC) was generated by varying the threshold value from the range −2 to +8.5 to find the optimum threshold value. FIG. 4 depicts a receiver operating curve (ROC) showing prediction performance of a method for scoring variants in an exome to predict an effect of the variants on gene, using a Clinvar database, in accordance with an embodiment of the present disclosure. An area under curve (AUC) value was obtained as 0.92 according to the ROC. The threshold value of 2.5 gives most optimum true positive rate (TPR) value of 0.90 and optimum false positive rate (FPR) value of 0.18 with highest accuracy. The TPR value of 0.85 and the corresponding FPR value of 0.13 was achieved with the change in the threshold value to 3.

To find the accuracy of the disclosed method, a comparison study was performed using the corresponding DANN value, utilizing the same dataset from Clinvar database used to generate the ROC using proposed method of scoring the variants. The ROC was generated by varying the threshold value from 0 to 1.1 of the DANN value corresponding to the variants present in Clinvar database. The AUC value obtained was 0.81. The threshold value 0.9 gives relatively balanced TPR and FPR values as 0.88 and 0.25 respectively. FIG. 5 depicts a receiver operating curve (ROC) using a deleterious annotation of genetic variants using neural networks (DANN) value corresponding to the variants present in a Clinvar database, in accordance with an embodiment of the present disclosure.

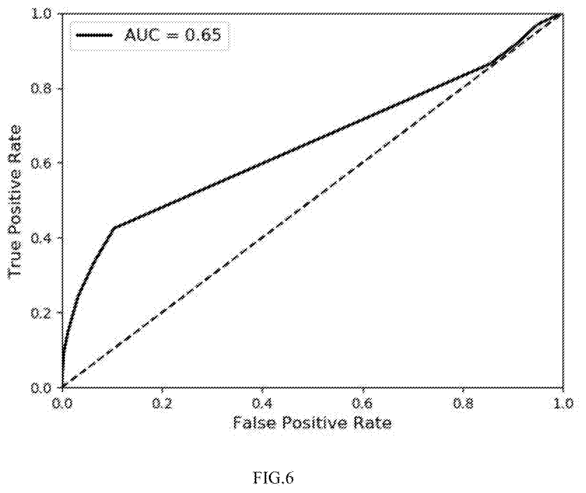

Another comparison study was performed using the corresponding FATHMM value corresponding to the non-synonymous variants, present in the Clinvar database. The ROC was generated by varying the threshold value from −10 to +10.64 of the FATHMM value. The AUC value obtained was 0.65. The threshold value of ‘−1’ gives relatively balanced TPR and FPR values as 0.42 and 0.10 respectively. FIG. 6 depicts a receiver operating curve (ROC) using a functional analysis through hidden markov models (FATHMM) value corresponding to the non-synonymous variants present in a Clinvar database, in accordance with an embodiment of the present disclosure.

A proper threshold value demonstrates a unique combination of high TPR and low FPR for variants. A high TPR is very much crucial in clinical interpretation because pathogenic variants should not be discarded falsely. On the other hand, having a low FPR means that the results is less contaminated with false positives and thus lower risk for samples being given a wrong molecular diagnosis. Hence we applied both the threshold values to check any difference in the prediction accuracy.

Table. 1 shows summary of the prediction performance of the disclosed method 200 on 1000 sample data from 1000Genome database for 78 metabolic disorder genes and 272 primary immunodeficiency genes.

TABLE 1

| | Number of healthy samples predicted as unhealthy | |
|---|---|---|
| | based on presence of one or more deleterious | |
| | mutations in one or both copy of the genes | |
| Gene | Threshold value (2.5) | Threshold value (3) |
| 78 Metabolic | 31 | 31 |
| disorder genes | | |
| 272 Primary | 328 | 328 |
| Immunodeficiency | | |
| genes | | |

According to the Table. 1, the threshold value of 2.5 was applied to the allele scores of 272 immunodeficiency genes, 328 among 1000 healthy samples in 1000 Genome are predicted to be containing at least one variant in homozygous state or two variants in trans or one variant in haploinsufficient gene with the final score more than to equal to 2.5 in any of the immunodeficiency genes. If the threshold value is 3, then the number of samples that are predicted unhealthy remain the same. Using the same criteria for 78 metabolic disorder genes, 31 samples out of 1000 samples in 1000 genome database are predicted to be containing at least one variant in homozygous state or at least two variants in trans or one variant in haploinsufficient gene with the final score more than to equal to 2.5. The number remain the same when the threshold value is increased to 3 from 2.5.

It was observed that when the threshold value of 2.5 used as for the variant to be deleterious, then the minimum score required to interpret the gene at risk should be 5 for haplosufficient gene and for haploinsufficient gene the minimum score to interpret the gene at risk is 2.5 and the sample is said to be containing at least one risk gene in the exome. Similarly, if the threshold value is 3, then any sample having the final score equal to or greater than 6 is a haplosufficient gene and the threshold value of 3 for haploinsufficient gene was predicted to contain at least one risk gene.

It is to be understood that the scope of the protection is extended to such a program and in addition to a computer-readable means having a message therein; such computer-readable storage means contain program-code means for implementation of one or more steps of the method, when the program runs on a server or mobile device or any suitable programmable device. The hardware device can be any kind of device which can be programmed including e.g. any kind of computer like a server or a personal computer, or the like, or any combination thereof. The device may also include means which could be e.g. hardware means like e.g. an application-specific integrated circuit (ASIC), a field-programmable gate array (FPGA), or a combination of hardware and software means, e.g. an ASIC and an FPGA, or at least one microprocessor and at least one memory with software processing components located therein. Thus, the means can include both hardware means and software means. The method embodiments described herein could be implemented in hardware and software. The device may also include software means. Alternatively, the embodiments may be implemented on different hardware devices, e.g. using a plurality of CPUs.

The embodiments herein can comprise hardware and software elements. The embodiments that are implemented in software include but are not limited to, firmware, resident software, microcode, etc. The functions performed by various components described herein may be implemented in other components or combinations of other components. For the purposes of this description, a computer-usable or computer readable medium can be any apparatus that can comprise, store, communicate, propagate, or transport the program for use by or in connection with the instruction execution system, apparatus, or device.

The illustrated steps are set out to explain the exemplary embodiments shown, and it should be anticipated that ongoing technological development will change the manner in which particular functions are performed. These examples are presented herein for purposes of illustration, and not limitation. Further, the boundaries of the functional building blocks have been arbitrarily defined herein for the convenience of the description. Alternative boundaries can be defined so long as the specified functions and relationships thereof are appropriately performed. Alternatives (including equivalents, extensions, variations, deviations, etc., of those described herein) will be apparent to persons skilled in the relevant art(s) based on the teachings contained herein. Such alternatives fall within the scope of the disclosed embodiments. Also, the words “comprising,” “having,” “containing,” and “including,” and other similar forms are intended to be equivalent in meaning and be open ended in that an item or items following any one of these words is not meant to be an exhaustive listing of such item or items, or meant to be limited to only the listed item or items. It must also be noted that as used herein and in the appended claims, the singular forms “a,” “an,” and “the” include plural references unless the context clearly dictates otherwise.

Furthermore, one or more computer-readable storage media may be utilized in implementing embodiments consistent with the present disclosure. A computer-readable storage medium refers to any type of physical memory on which information or data readable by a processor may be stored. Thus, a computer-readable storage medium may store instructions for execution by one or more processors, including instructions for causing the processor(s) to perform steps or stages consistent with the embodiments described herein. The term “computer-readable medium” should be understood to include tangible items and exclude carrier waves and transient signals, i.e., be non-transitory. Examples include random access memory (RAM), read-only memory (ROM), volatile memory, nonvolatile memory, hard drives, CD ROMs, DVDs, flash drives, disks, and any other known physical storage media.

It is intended that the disclosure and examples be considered as exemplary only, with a true scope of disclosed embodiments being indicated by the following claims.

What is claimed is:

1. A processor-implemented method for scoring variants in an exome to predict an effect of the variants on gene function, the method comprising the steps of:
receiving, via the one or more hardware processors, a dataset comprising a plurality of variants corresponding to the exome, wherein the plurality of variants are one or more single nucleotide variants (SNVs) and one or more indels;
annotating, via the one or more hardware processors, each of the plurality of variants comprised in the dataset with corresponding variant information, to form a plurality of annotated variants;
identifying, via the one or more hardware processors, one or more variants, out of the plurality of annotated variants, occurring in a transcript of a plurality of transcripts corresponding to a protein coding gene comprised in the exome, to form a set of variants, wherein the one or more variants are identified based on a corresponding transcript ID;
separating, via the one or more hardware processors, variants in a Y-chromosome from the set of variants, to form a revised set of variants;
identifying, via the one or more hardware processors, (i) one or more SNVs present in the coding exonic region and a coding intronic region, and one or more indels present in the coding intronic region, based on a corresponding minor allele frequency (MAF) value, and (ii) one or more indels present in a coding exonic region, from the revised set of variants, to form a subset of variants;
assessing, via the one or more hardware processors, the identified one or more SNVs and the identified one or more indels from the subset of variants, wherein assessing the identified one or more SNVs comprises (i) selecting the one or more SNVs based on a corresponding ethnicity wise allele frequency (ETH_AF) value, from the identified one or more SNVs, and (ii) assigning a score for each of the selected one or more SNVs, based on (i) presence in the coding exonic region and (ii) presence in the coding intronic region, and wherein assessing the identified one or more indels comprises assigning the score for each of the identified one or more indels, based on (i) presence in a coding exonic intronic boundary region (ii) presence in the coding exonic region, and (iii) presence in the coding intronic region;
assigning, via the one or more hardware processors, a final score for each of the selected one or more SNVs and the identified one or more indels, based on the corresponding assigned score, a corresponding genomic evolutionary rate profiling (Gerp)++ RSbase value and a corresponding sub-region residual variation intolerance scores (SubRVIS) value; and
predicting, via the one or more hardware processors, the effect of the one or more variants on the gene function, based on the corresponding final score, corresponding genotype information and haploinsufficiency of the gene.

2. The method of claim 1, wherein each variant of the plurality of variants comprises a corresponding chromosome number, a corresponding genomic position, a corresponding reference allele, a corresponding alternative allele, and the corresponding genotype information.

3. The method of claim 1, wherein the corresponding variant information of each variant of the plurality of variants comprising one or more of: a corresponding gene name, the corresponding subRVIS value, the corresponding minor allele frequency (MAF) value, the corresponding ethnicity wise allele frequency (ETH_AF) value, a corresponding region of the variant, the corresponding transcript ID, a corresponding mutation type, corresponding information related to change in amino-acid, the corresponding Gerp++ RSbase value, the corresponding dbScSNV values comprising a corresponding adaboost (Ada) value and a corresponding random forest (RF) value, a corresponding deleterious annotation of genetic variants using neural networks (DANN) value, a corresponding sorting intolerant from tolerant (SIFT) value, a corresponding protein variation effect analyzer (PROVEAN) value, a corresponding functional analysis through hidden markov models (FATHMM) value, a corresponding mendelian clinically applicable pathogenicity (M-CAP) value, and a corresponding meta-analytic support vector machine (MetaSVM) value.

4. The method of claim 1, wherein assigning the score for each of the selected one or more SNVs present in the coding exonic region, comprising:
categorizing the selected one or more SNVs into: (i) coding exonic splice region SNVs and (ii) coding exonic non-splice region SNVs, wherein the coding exonic splice region SNVs are the selected one or more SNVs that fall under a splice region and the coding exonic non-splice region SNVs are the selected one or more SNVs that does not fall under the splice region;
assigning an initial score to the coding exonic non-splice region SNVs;
assigning initial scores to the coding exonic splice region SNVs, based on the corresponding Ada value and the corresponding RF value;
sub-categorizing the coding exonic splice region SNVs and the coding exonic non-splice region SNVs into: (i) non-synonymous SNVs group (ii) synonymous SNVs group and (iii) gain-loss mutation SNVs group, based on the corresponding mutation type, wherein the gain-loss mutation SNVs group includes stop gain mutation SNVs, stop loss mutation SNVs, start gain mutation SNVs and start loss mutation SNVs;
assigning the score for each of the coding exonic splice region SNVs and each of the coding exonic non-splice region SNVs, comprised in the non-synonymous SNVs group, based on (i) the corresponding initial score, (ii) outcome of SNVs deleteriousness prediction tools, and (iii) a change in amino acid within predefined amino acid groups and an outcome of SNVs protein function effect prediction tool;
assigning the score for each of the coding exonic splice region SNVs and each of the coding exonic non-splice region SNVs, comprised in the synonymous SNVs group, based on (i) the corresponding initial score and (ii) the outcome of SNVs deleteriousness prediction tool; and
assigning the score for each of the coding exonic splice region SNVs and each of the coding exonic non-splice region SNVs, comprised in the gain-loss mutation SNVs group, based on (i) the corresponding initial score and (ii) the outcome of SNVs deleteriousness prediction tool.

5. The method of claim 1, wherein assigning the score for each of the identified one or more indels present in the coding exonic region, comprising:
categorizing the identified one or more indels present in the coding exonic region into (i) a non-frameshift indels group and (ii) a frameshift indels group, based on the corresponding mutation type;
assigning the score for each of the identified one or more indels comprised in the non-frameshift indels group, based on (i) the corresponding MAF value (ii) the corresponding ETH_AF value and (iii) the outcome of indels deleteriousness prediction tool; and
assigning the score for each of the identified one or more indels comprised in the frameshift indels group, comprising:
categorizing the identified one or more indels into one or more deletion indels and one or more insertion indels, based on a length of the corresponding reference allele (len_ref) and a length of the corresponding altered allele (len_alt);
calculating an insertion length of each of the one or more insertion indels and a deletion length (del_len) of each of the one or more deletion indels, based on the corresponding len_ref and the corresponding len_alt;
calculating a haplo1_indel value as a sum of insertions occurring in haplotype1 (haplo1_ins value) and deletions occurring in haplotype1 (haplo1_del value), and a haplo2_indel value as sum of the insertions occurring in haplotype2 (haplo2_ins value) and the deletions occurring in haplotype2 (haplo2_del value), haplotype1 (h1) represent one gene copy and haplotype2 (h2) represent the another gene copy, wherein the haplo1_ins value is a total length of the one or more insertion indels present in the haplotype1 (h1), the haplo1_del value is a total length of the one or more deletion indels present in the haplotype1 (h1), and the haplo2_ins value is a total length of the one or more insertion indels present in the haplotype2 (h2), the haplo2_del value is a total length of the one or more deletion indels present in the haplotype2 (h2);
calculating a haplotype1_score based on a change in reading frame of the gene in haplotype1 (h1) and a h1_count and a haplotype2_score based on a change in reading frame of the gene in haplotype2 (h2) and a h2_count, wherein the h1_count is calculated based on a number of indels present in the haplotype1 (h1) and the number of indels present in the haplotype1 (h1) having the MAF value greater than the predefined Th_MAF value, and the h2_count is calculated based on the number of indels present in the haplotype2 (h2) and the number of indels present in the haplotype2 (h2) having the MAF value greater than the predefined Th_MAF value; and
assigning the score for each of the identified one or more indels based on a h1_allele score and a h2 allele score, wherein the h1_allele score is calculated based on the haplotype1_score and the h1_count, and the h2_allele score is calculated based on the haplotype2_score and the h2_count.

6. The method of claim 1, wherein assigning the score for each of the identified one or more indels present in the coding exonic intronic boundary region, comprising:
selecting the one or more indels from the identified one or more indels, based on the corresponding MAF value less than the predefined threshold value;
categorizing the selected one or more indels into insertion indels and deletion indels, based on a length of the corresponding reference allele (len_ref) and a length of the corresponding altered allele (len_alt);
sub-categorizing the insertion indels into donor insertion indels and acceptor insertion indels, and the deletion indels into donor deletion indels and acceptor deletion indels, based on the corresponding genomic position;
assigning the score for each of the donor deletion indels, by:
calculating a MaxEnt value for a plurality of donor consensus (GTs) present between −50 bp and +50 bp from a position of the corresponding donor deletion indel to identify the donor consensus having the maximum MaxEnt value from the plurality of donor consensus (GTs); and
assigning the score for the corresponding donor deletion indel based on a change in a exon length, considering the identified donor consensus having the maximum MaxEnt value as a cryptic donor GT;
assigning the score for each of the acceptor deletion indels, by:
calculating the MaxEnt value for a plurality of acceptor consensus (AGs) present between −50 bp and +50 bp from the position of the corresponding acceptor deletion indel to identify the acceptor consensus having the maximum MaxEnt value from the plurality of the acceptor consensus (AGs); and
assigning the score for the corresponding acceptor deletion indel based on the change in the exon length, considering the identified acceptor consensus having the maximum MaxEnt value as a cryptic acceptor AG;
assigning the score for each of the donor insertion indels based on: (i) the corresponding donor insertion indel generating or not generating a new donor consensus, (ii) the MaxEnt value of the new donor consensus and the MaxEnt value of the natural donor consensus in mutated sequence, and (iii) the MaxEnt value of the new donor consensus, the MaxEnt value of the natural donor consensus in wildtype sequence and the change in the exon length; and
assigning the score for each of the acceptor insertion indels based on: (i) the corresponding acceptor insertion indel generating or not generating a new acceptor consensus, (ii) the MaxEnt value of the new acceptor consensus and the MaxEnt value of the natural acceptor consensus in mutated sequence, and (iii) the MaxEnt value of the new acceptor consensus, the MaxEnt value of the natural acceptor consensus in wildtype sequence and the change in the exon length.

7. The method of claim 1, wherein assigning the score for each of the identified one or more indels and the selected one or more SNVs present in the coding intronic region, comprising:
categorizing the identified one or more indels and the selected one or more SNVs present in the coding intronic region into (i) donor coding intronic variants and (ii) acceptor coding intronic variants, based on the corresponding genomic position;
assigning the score for each of the donor coding intronic variants and the acceptor coding intronic variants, wherein,
assigning the score for each of the donor coding intronic variants, based on: (i) the variant having a natural donor site disrupted or weakened or not affected (ii) the MaxEnt value of the natural donor site, if the variant with natural donor site not disrupted, (iii) the MaxEnt value of the cryptic donor site, if the cryptic donor site is generated, and (iv) a position of natural donor site and the position of the cryptic donor site;
assigning the score for each of the acceptor coding intronic variants, based on the corresponding position of the variant (pos_var) from the acceptor site, wherein:
assigning the score for each of the acceptor coding intronic variants having the pos_var less than 15, based on: (i) the variant with the natural acceptor site disrupted or weakened or not affected, (ii) the MaxEnt value of the natural acceptor site, if the variant with natural acceptor site not disrupted, (iii) the MaxEnt value of the cryptic acceptor site, if the cryptic acceptor site is generated, and (iv) a position of natural acceptor site and the position of the cryptic acceptor site;
assigning the score for each of the acceptor coding intronic variants having the pos_var between 15 and 20, based on: (i) the variant causing the branch point disruption, and (ii) the variant not causing the branch point disruption, wherein,
the score for the variant causing the branch point disruption is assigned based on a presence of an existing compensating branch point or a newly created compensating branch point; and
the score for the variant not causing the branch point disruption is assigned based on at least one of (i) the natural acceptor site weakened or not weakened (ii) the MaxEnt value of natural acceptor site, (iii) the MaxEnt value of cryptic acceptor site if the cryptic acceptor site is generated (iv) the position of natural acceptor site and the position of the cryptic acceptor site;
assigning the score for each of the acceptor coding intronic variants having the pos_var between 21 and 49, based on at least one of: (i) branch point disrupted or not disrupted (ii) presence of an existing compensating branch point (iii) a newly created branch point; and
assigning the score for each of the acceptor coding intronic variants having the pos_var 50 or more, with the predefined value.

8. A system for scoring variants in an exome to predict an effect of the variants on gene function, the system comprising:
a memory storing instructions;
one or more communication interfaces; and
one or more hardware processors coupled to the memory via the one or more communication interfaces, wherein the one or more hardware processors are configured by the instructions to:
receive a dataset comprising a plurality of variants corresponding to the exome, wherein the plurality of variants are one or more single nucleotide variants (SNVs) and one or more indels;
annotate each of the plurality of variants comprised in the dataset with corresponding variant information, to form a plurality of annotated variants;
identify one or more variants, out of the plurality of annotated variants, occurring in a transcript of a plurality of transcripts corresponding to a protein coding gene comprised in the exome, to form a set of variants, wherein the one or more variants are identified based on a corresponding transcript ID;
separate variants in a Y-chromosome from the set of variants, to form a revised set of variants;
identify (i) one or more SNVs present in the coding exonic region and a coding intronic region, and one or more indels present in the coding intronic region, based on a corresponding minor allele frequency (MAF) value, and (ii) one or more indels present in a coding exonic region, from the revised set of variants, to form a subset of variants;
assess the identified one or more SNVs and the identified one or more indels from the subset of variants, wherein assessing the identified one or more SNVs comprises (i) selecting the one or more SNVs based on a corresponding ethnicity wise allele frequency (ETH_AF) value, from the identified one or more SNVs, and (ii) assigning a score for each of the selected one or more SNVs, based on (i) presence in the coding exonic region and (ii) presence in the coding intronic region, and wherein assessing the identified one or more indels comprises assigning the score for each of the identified one or more indels, based on (i) presence in a coding exonic intronic boundary region (ii) presence in the coding exonic region, and (iii) presence in the coding intronic region;
assign a final score for each of the selected one or more SNVs and the identified one or more indels, based on the corresponding assigned score, a corresponding genomic evolutionary rate profiling (Gerp)++ RSbase value and a corresponding sub-region residual variation intolerance scores (SubRVIS) value; and
predict the effect of the one or more variants on the gene function, based on the corresponding final score, corresponding genotype information and haploinsufficiency of the gene.

9. The system of claim 8, wherein each variant of the plurality of variants comprises a corresponding chromosome number, a corresponding genomic position, a corresponding reference allele, a corresponding alternative allele, and the corresponding genotype information.

10. The system of claim 8, wherein the corresponding variant information of each variant of the plurality of variants comprising one or more of: a corresponding gene name, the corresponding subRVIS value, the corresponding minor allele frequency (MAF) value, the corresponding ethnicity wise allele frequency (ETH_AF) value, a corresponding region of the variant, the corresponding transcript ID, a corresponding mutation type, corresponding information related to change in amino-acid, the corresponding Gerp++ RSbase value, the corresponding dbScSNV values comprising a corresponding adaboost (Ada) value and a corresponding random forest (RF) value, a corresponding deleterious annotation of genetic variants using neural networks (DANN) value, a corresponding sorting intolerant from tolerant (SIFT) value, a corresponding protein variation effect analyzer (PROVEAN) value, a corresponding functional analysis through hidden markov models (FATHMM) value, a corresponding mendelian clinically applicable pathogenicity (M-CAP) value, and a corresponding meta-analytic support vector machine (MetaSVM) value.

11. The system of claim 8, wherein the one or more hardware processors are configured to assign the score for each of the selected one or more SNVs present in the coding exonic region, by:
categorizing the selected one or more SNVs into: (i) coding exonic splice region SNVs and (ii) coding exonic non-splice region SNVs, wherein the coding exonic splice region SNVs are the selected one or more SNVs that fall under a splice region and the coding exonic non-splice region SNVs are the selected one or more SNVs that does not fall under the splice region;
assigning an initial score to the coding exonic non-splice region SNVs;
assigning initial scores to the coding exonic splice region SNVs, based on the corresponding Ada value and the corresponding RF value;
sub-categorizing the coding exonic splice region SNVs and the coding exonic non-splice region SNVs into: (i) non-synonymous SNVs group (ii) synonymous SNVs group and (iii) gain-loss mutation SNVs group, based on the corresponding mutation type, wherein the gain-loss mutation SNVs group includes stop gain mutation SNVs, stop loss mutation SNVs, start gain mutation SNVs and start loss mutation SNVs;
assigning the score for each of the coding exonic splice region SNVs and each of the coding exonic non-splice region SNVs, comprised in the non-synonymous SNVs group, based on (i) the corresponding initial score, (ii) outcome of SNVs deleteriousness prediction tools, and (iii) a change in amino acid within predefined amino acid groups and an outcome of SNVs protein function effect prediction tool;
assigning the score for each of the coding exonic splice region SNVs and each of the coding exonic non-splice region SNVs, comprised in the synonymous SNVs group, based on (i) the corresponding initial score and (ii) the outcome of SNVs deleteriousness prediction tool; and
assigning the score for each of the coding exonic splice region SNVs and each of the coding exonic non-splice region SNVs, comprised in the gain-loss mutation SNVs group, based on (i) the corresponding initial score and (ii) the outcome of SNVs deleteriousness prediction tool.

12. The system of claim 8, wherein the one or more hardware processors are configured to assign the score for each of the identified one or more indels present in the coding exonic region, by:
categorizing the identified one or more indels present in the coding exonic region into (i) a non-frameshift indels group and (ii) a frameshift indels group, based on the corresponding mutation type;
assigning the score for each of the identified one or more indels comprised in the non-frameshift indels group, based on (i) the corresponding MAF value (ii) the corresponding ETH_AF value and (iii) the outcome of indels deleteriousness prediction tool; and
assigning the score for each of the identified one or more indels comprised in the frameshift indels group, comprising:
categorizing the identified one or more indels into one or more deletion indels and one or more insertion indels, based on a length of the corresponding reference allele (len_ref) and a length of the corresponding altered allele (len_alt);
calculating an insertion length of each of the one or more insertion indels and a deletion length (del_len) of each of the one or more deletion indels, based on the corresponding len_ref and the corresponding len_alt;
calculating a haplo1_indel value as a sum of insertions occurring in haplotype1 (haplo1_ins value) and deletions occurring in haplotype1 (haplo1_del value), and a haplo2_indel value as sum of the insertions occurring in haplotype2 (haplo2_ins value) and the deletions occurring in haplotype2 (haplo2_del value), haplotype1 (h1) represent one gene copy and haplotype2 (h2) represent the another gene copy, wherein the haplo1_ins value is a total length of the one or more insertion indels present in the haplotype1 (h1), the haplo1_del value is a total length of the one or more deletion indels present in the haplotype1 (h1), and the haplo2_ins value is a total length of the one or more insertion indels present in the haplotype2 (h2), the haplo2_del value is a total length of the one or more deletion indels present in the haplotype2 (h2);
calculating a haplotype1_sore based on a change in reading frame of the gene in haplotype1 (h1) and a h1_count and a haplotype2_score based on a change in reading frame of the gene in haplotype2 (h2) and a h2_count, wherein the h_count is calculated based on a number of indels present in the haplotype1 (h1) and the number of indels present in the haplotype1 (h1) having the MAF value greater than the predefined Th_MAF value, and the h2_count is calculated based on the number of indels present in the haplotype2 (h2) and the number of indels present in the haplotype2 (h2) having the MAF value greater than the predefined Th_MAF value; and
assigning the score for each of the identified one or more indels based on a h1_allele score and a h2_allele score, wherein the h1_allele score is calculated based on the haplotype1_score and the h1_count, and the h2_allele score is calculated based on the haplotype2_score and the h2_ount.

13. The system of claim 8, wherein the one or more hardware processors are configured to assign the score for each of the identified one or more indels present in the coding exonic intronic boundary region, by:
selecting the one or more indels from the identified one or more indels, based on the corresponding MAF value less than the predefined threshold value;
categorizing the selected one or more indels into insertion indels and deletion indels, based on a length of the corresponding reference allele (len_ref) and a length of the corresponding altered allele (len_alt);
sub-categorizing the insertion indels into donor insertion indels and acceptor insertion indels, and the deletion indels into donor deletion indels and acceptor deletion indels, based on the corresponding genomic position;
assigning the score for each of the donor deletion indels, by:
calculating a MaxEnt value for a plurality of donor consensus (GTs) present between −50 bp and +50 bp from a position of the corresponding donor deletion indel to identify the donor consensus having the maximum MaxEnt value from the plurality of donor consensus (GTs); and
assigning the score for the corresponding donor deletion indel based on a change in a exon length, considering the identified donor consensus having the maximum MaxEnt value as a cryptic donor GT;
assigning the score for each of the acceptor deletion indels, by:
calculating the MaxEnt value for a plurality of acceptor consensus (AGs) present between −50 bp and +50 bp from the position of the corresponding acceptor deletion indel to identify the acceptor consensus having the maximum MaxEnt value from the plurality of the acceptor consensus (AGs); and
assigning the score for the corresponding acceptor deletion indel based on the change in the exon length, considering the identified acceptor consensus having the maximum MaxEnt value as a cryptic acceptor AG;
assigning the score for each of the donor insertion indels based on: (i) the corresponding donor insertion indel generating or not generating a new donor consensus, (ii) the MaxEnt value of the new donor consensus and the MaxEnt value of the natural donor consensus in mutated sequence, and (iii) the MaxEnt value of the new donor consensus, the MaxEnt value of the natural donor consensus in wildtype sequence and the change in the exon length; and
assigning the score for each of the acceptor insertion indels based on: (i) the corresponding acceptor insertion indel generating or not generating a new acceptor consensus, (ii) the MaxEnt value of the new acceptor consensus and the MaxEnt value of the natural acceptor consensus in mutated sequence, and (iii) the MaxEnt value of the new acceptor consensus, the MaxEnt value of the natural acceptor consensus in wildtype sequence and the change in the exon length.

14. The system of claim 8, wherein the one or more hardware processors are configured to assign the score for each of the identified one or more indels and the selected one or more SNVs present in the coding intronic region, by:
categorizing the identified one or more indels and the selected one or more SNVs present in the coding intronic region into (i) donor coding intronic variants and (ii) acceptor coding intronic variants, based on the corresponding genomic position;
assigning the score for each of the donor coding intronic variants and the acceptor coding intronic variants, wherein,
assigning the score for each of the donor coding intronic variants, based on: (i) the variant having a natural donor site disrupted or weakened or not affected (ii) the MaxEnt value of the natural donor site, if the variant with natural donor site not disrupted, (iii) the MaxEnt value of the cryptic donor site, if the cryptic donor site is generated, and (iv) a position of natural donor site and the position of the cryptic donor site;
assigning the score for each of the acceptor coding intronic variants, based on the corresponding position of the variant (pos_var) from the acceptor site, wherein:
assigning the score for each of the acceptor coding intronic variants having the pos_var less than 15, based on: (i) the variant with the natural acceptor site disrupted or weakened or not affected, (ii) the MaxEnt value of the natural acceptor site, if the variant with natural acceptor site not disrupted, (iii) the MaxEnt value of the cryptic acceptor site, if the cryptic acceptor site is generated, and (iv) a position of natural acceptor site and the position of the cryptic acceptor site;
assigning the score for each of the acceptor coding intronic variants having the pos_var between 15 and 20, based on: (i) the variant causing the branch point disruption, and (ii) the variant not causing the branch point disruption, wherein,
the score for the variant causing the branch point disruption is assigned based on a presence of an existing compensating branch point or a newly created compensating branch point; and
the score for the variant not causing the branch point disruption is assigned based on at least one of (i) the natural acceptor site weakened or not weakened (ii) the MaxEnt value of natural acceptor site, (iii) the MaxEnt value of cryptic acceptor site if the cryptic acceptor site is generated (iv) the position of natural acceptor site and the position of the cryptic acceptor site;
assigning the score for each of the acceptor coding intronic variants having the pos_var between 21 and 49, based on at least one of: (i) branch point disrupted or not disrupted (ii) presence of an existing compensating branch point (iii) a newly created branch point; and
assigning the score for each of the acceptor coding intronic variants having the pos_var 50 or more, with the predefined value.

15. A computer program product comprising a non-transitory computer readable medium having a computer readable program embodied therein, wherein the computer readable program, when executed on a computing device, causes the computing device to:
receive a dataset comprising a plurality of variants corresponding to the exome, wherein the plurality of variants are one or more single nucleotide variants (SNVs) and one or more indels;
annotate each of the plurality of variants comprised in the dataset with corresponding variant information, to form a plurality of annotated variants;
identify one or more variants, out of the plurality of annotated variants, occurring in a transcript of a plurality of transcripts corresponding to a protein coding gene comprised in the exome, to form a set of variants, wherein the one or more variants are identified based on a corresponding transcript ID;
separate variants in a Y-chromosome from the set of variants, to form a revised set of variants;
identify (i) one or more SNVs present in the coding exonic region and a coding intronic region, and one or more indels present in the coding intronic region, based on a corresponding minor allele frequency (MAF) value, and (ii) one or more indels present in a coding exonic region, from the revised set of variants, to form a subset of variants;
assess the identified one or more SNVs and the identified one or more indels from the subset of variants, wherein assessing the identified one or more SNVs comprises (i) selecting the one or more SNVs based on a corresponding ethnicity wise allele frequency (ETH_AF) value, from the identified one or more SNVs, and (ii) assigning a score for each of the selected one or more SNVs, based on (i) presence in the coding exonic region and (ii) presence in the coding intronic region, and wherein assessing the identified one or more indels comprises assigning the score for each of the identified one or more indels, based on (i) presence in a coding exonic intronic boundary region (ii) presence in the coding exonic region, and (iii) presence in the coding intronic region;
assign a final score for each of the selected one or more SNVs and the identified one or more indels, based on the corresponding assigned score, a corresponding genomic evolutionary rate profiling (Gerp)++ RSbase value and a corresponding sub-region residual variation intolerance scores (SubRVIS) value; and
predict the effect of the one or more variants on the gene function, based on the corresponding final score, corresponding genotype information and haploinsufficiency of the gene.
